(19) United States
(12) Patent Application Publication
(10) Pub. No.: US 2009/0299213 A1
(43) Pub. Date: Dec. 3, 2009

(54) Nanobioelectronics

(75) Inventors: Fernando Patolsky, Rehovot (IL); Brian P. Timko, Cambridge, MA (US); Guihua Yu, Somerville, MA (US); Charles M. Lieber, Lexington, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 12/225,142

(22) PCT Filed: Mar. 15, 2007
(86) PCT No.: PCT/US07/06545

§ 371 Date: Mar. 11, 2009

Related U.S. Application Data
(60) Provisional application No. 60/783,203, filed on Mar. 15, 2006.

(51) Int. Cl. A61B 5/05; H01L 29/66

(52) U.S. Cl. 600/554; 257/14; 257/E29.168; 977/925

(57) ABSTRACT

The present invention generally relates to nanobioelectronics and, in some cases, to circuits comprising nanoelectronic elements, such as nanotubes and/or nanowires, and biological components, such as neurons. In one aspect, cells, such as neurons, are positioned in electrical communication with one or more nanoscale wires. The nanoscale wires may be used to stimulate the cells, and/or determine an electrical condition of the cells. More than one nanoscale wire may be positioned in electrical communication with the cell, for example, in distinct regions of the cell. However, the nanoscale wires may be positioned such that they are relatively close together, for example, spaced apart by no more than about 200 nm. The nanoscale wires may be disposed on a substrate, for example, between electrodes, and the cells may be adhered to the substrate, for example, using cell adhesion factors such as polylysine. Also provided in other aspects of the invention are methods for making and using such devices, kits for using the same, and the like.

### RELATED APPLICATIONS

This application claims the benefit of U.S. Provisional Patent Application Ser. No. 60/783,203, filed Mar. 15, 2006, entitled “Nanobioelectronics,” by Patolsky, et al.

### GOVERNMENT FUNDING

Research leading to various aspects of the present invention were sponsored, at least in part, by DARPA. The U.S. Government may have certain rights in the invention.

### FIELD OF INVENTION

The present invention generally relates to nanobioelectronics and, in some cases, to circuits comprising nanoelectronic elements, such as nanotubes and/or nanowires, and biological components, such as neurons.

### BACKGROUND

Electrophysiological measurements made using micropipette electrodes and microfabricated electrode arrays play an important role in understanding signal propagation through individual neurons and neuronal networks. Micropipette electrodes can stimulate and record intracellular and extracellular potentials in vitro and in vivo with relatively good spatial resolution, yet are difficult to multiplex. Microfabricated structures, such as electrode arrays, have enabled multiplexed recording from relatively large numbers of electrodes needed for investigating networks, but lack the resolution necessary to provide fine-grain information at the level of individual cells.

### SUMMARY OF THE INVENTION

The present invention generally relates to nanobioelectronics and, in some cases, to circuits comprising nanoelectronic elements, such as nanotubes and/or nanowires, and biological, components, such as neurons. The subject matter of the present invention involves, in some cases, interrelated products, alternative solutions to a particular problem, and/or a plurality of different uses of one or more systems and/or articles.

Various aspects of the invention are recited in the claims which accompany this application.

In one aspect, the invention is directed to an article. In one set of embodiments, the article includes a nanoscale wire, and a cell in electrical communication with the nanoscale wire. The article, in another set of embodiments, includes a cell, a first electrode in electrical communication with the cell, and a second electrode in electrical communication with the cell. In some cases, the first electrode and the second electrode are separated by a distance of no more than about 200 nm. The article, in yet another set of embodiments, includes a surface of a substrate, a plurality of nanoscale wires substantially parallel on the substrate, and a cell adhesion factor deposited on at least a portion of the substrate.

In one set of embodiments, the article includes a first electrical connector, a second electrical connector, and a nanoscale wire in physical contact with both the first electrical connector and the second electrical connector, and a cell in physical contact with the nanoscale wire. The article, in another set of embodiments, includes a cell, and at least 3 electrodes, each in electrical communication with the cell, each electrode independently measuring a distinct region of the cell. According to still another set of embodiments, the article includes a cell, a first electrode comprising a p-type material in electrical communication with the cell, and a second electrode comprising an n-type material in electrical communication with the cell. In one set of embodiments, the article includes a logic gate that can be deactivated upon exposure to a neurotoxin.

Another aspect of the invention is directed to a method. The method, in one set of embodiments, includes an act of passing electrical current through a nanoscale wire in physical contact with a cell. In another set of embodiment, the method includes an act of determining an electrical state of a cell using a nanoscale wire. The method, in yet another set of embodiments, includes an act of depositing cell adhesion factor on a substrate comprising nanoscale wires.

In another aspect, the present invention is directed to a method of making one or more of the embodiments described herein, for example, an article comprising a nanoscale wire, and a cell in electrical communication with the nanoscale wire. In another aspect, the present invention is directed to a method of using one or more of the embodiments described herein, for example, an article comprising a nanoscale wire, and a cell in electrical communication with the nanoscale wire.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1P illustrate the recording of certain neuronal axon signals, according to one embodiment of the invention;

FIGS. 2A-2F illustrate certain multi-nanowire/neuron arrays, according to another embodiment of the invention;

FIGS. 3A-3D illustrate certain multi-nanowire/neurite hybrid structures, according to yet another embodiment of the invention;

FIGS. 4A-4D illustrate hybrid nanowire/neuron circuits and logic gates, according to still another embodiment of the invention;

FIGS. 5A-5D illustrate integrated nanowire/neuron devices, according to yet another embodiment of the invention;

FIGS. 6A-6G illustrate certain nanowire/axon devices, in yet another embodiment of the invention;

FIGS. 7A-7I illustrate nanowire detection of signals, in still another embodiment of the invention;

FIGS. 8A-8C illustrate nanowire stimulation of neurons, in yet another embodiment of the invention; and

FIGS. 9A-9D illustrate electrical and chemical modulation of signal propagation, according to still another embodiment of the invention.

### DETAILED DESCRIPTION

The present invention generally relates to nanobioelectronics and, in some cases, to circuits comprising nanoelectronic elements, such as nanotubes and/or nanowires, and biological components, such as neurons. In one aspect, cells, such as neurons, are positioned in electrical communication with one or more nanoscale wires. The nanoscale wires may be used to stimulate the cells, and/or determine an electrical condition of the cells. More than one nanoscale wire may be positioned in electrical communication with the cell, for example, in distinct regions of the cell. However, the nanoscale wires may be positioned such that they are relatively close together, for example, spaced apart by no more than about 200 nm. The nanoscale wires may be disposed on a substrate, for example, between electrodes, and the cells may be adhered to the substrate, for example, using cell adhesion factors such as polylysine. Also provided in other aspects of the invention are methods for making and using such devices, kits for using the same, and the like.

In one aspect of the invention, cells such as neurons are positioned in electrical communication with one or more nanoscale wires, for example, nanowires (e.g., semiconductor nanowires) and/or nanotubes, as described in detail below. Practically any cell can be used which exhibits electrical behavior, such as membrane potential. For instance, the cell may be a cell in which it is desired to measure the membrane potential (e.g., instantaneously, as a function of time, in response to an external stimulus, such as a drug or an applied external electrical potential, etc.), the cell may be a cell which can be used to detect electric fields (for example, cells from the ampullae of Lorenzini, which is present in certain types of organisms such as sharks), or the cell may be a cell that can produce an electrical signal, for example, a neuron (which is able to produce an action potential) or an electrocyte (which is used in organisms such as electric eels or electric ray to produce an electrical discharge).

The nanoscale wire may be in electrical communication with a portion of the cell, i.e., the nanoscale wire may be positioned, relative to the cell, such that the nanoscale wire is able to determine or affect the electrical behavior of the cell, and/or of a region of the cell. The nanoscale wires are typically of dimensions such that the nanoscale wire can be used to measure or determine a distinct region of a cell. As a non-limiting example, if the cell is a neuron, the nanoscale wire may be positioned such that the nanoscale wire is able to determine or affect the electrical behavior of a portion of the axon, dendrite, and/or soma of the neuron. The nanoscale may be in physical contact with the cell, or not in physical contact but positioned such that changes in the electrical state of the cell are able to affect the electrical state of the nanoscale wire, and/or vice versa.

In one set of embodiments, a cell in electrical communication with a nanoscale wire can be electrically stimulated by passing a current or applying a potential to the nanoscale wire, which may be used to affect the electrical state of the cell. For example, the membrane potential of a cell may be altered upon electrical stimulation, or a neuron can be stimulated to cause the neuron to polarize (e.g., hyperpolarize) or depolarize upon the application of sufficient current or potential. Additionally, in some cases, the electrical state of the cell can be determined using a sensing electrode, such as another nanoscale wire, as discussed below.

In another set of embodiments, a change in an electrical state of a cell, such as cell polarization or depolarization, an action potential, a change in plasma membrane potential, or the like may cause a change in the electrical state of a nanoscale wire in electrical communication with the cell, such as a change in conductance, which change can be determined and/or recorded in some fashion, e.g., using techniques known to those of ordinary skill in the art. Accordingly, one embodiment of the invention provides for the determination of an electrical state of a cell using a nanoscale wire. For example, if the nanoscale wire is part of a transistor, such as a field-effect transistor (FET), the electrical response of the cell to the change in electrical state may be determined by determining the state of the FET using techniques known to those of ordinary skill in the art. In some cases, the cell may also be one which was electrically stimulated, e.g., electrically stimulated by applying current or a potential to an electrode, such as another nanoscale wire, that is in electrical communication with the cell. As a specific example, the electrical state of a neuron, or a portion thereof (e.g., an axon, a dendrite, a soma, etc.) may be determined using a nanoscale wire in electrical communication with the neuron; for instance, the neuron may depolarize (e.g., due to exposure to a chemical species, or to a nanoscale wire or other electrode able to cause the neuron to depolarize), causing the formation and propagation of an action potential through the neuron, which action potential may be determined using a nanoscale wire.

In some embodiments, the electrical state of the cell may be altered by exposing the cell to a chemical species suspected of being able to alter the electrical state of the cell. For example, a chemical species able to facilitate the depolarization of a cell, or a chemical species that inhibit the depolarization of a cell, can be used to alter the electrical state of the cell, and in some cases, to cause a cell such as a neuron to polarize (e.g., hyperpolarize) or depolarize. In one set of embodiments, the chemical species comprises a neurotoxin, such as tetrodotoxin (which may block action potentials in nerves by binding to the pores of voltage-gated sodium channels) or batrachotoxin (which may affect the nervous system by causing depolarization due to increased sodium ion permeability). Such chemical species may, in some cases, deactivate or kill the cells, and in certain embodiments, e.g., if the cells are used as components of a device, the chemical species may inactivate the device.

Due to their small size, more than one electrode, e.g., comprising a nanoscale wire, may be positioned in electrical communication with the cell, or portion thereof, according to another set of embodiments. For example, at least 3, at least 4, at least 5, at least 10, at least 15, at least 20, at least 25, at least 30, at least 35, at least 40, at least 45, or at least 50 or more nanoscale wires may be positioned in electrical communication with the cell, or with a portion thereof, e.g., axons and/or dendrites if the cell is neuron. Thus, a plurality of nanoscale wires may each be used to independently measure a distinct region of the cell.

If more than one nanoscale wire is present, the nanoscale wires may each independently be the same or different. For example, the nanoscale wires may be doped or undoped, and/or may comprise p-type or n-type materials. For example, 1, 2, 3, or 4, etc. p-type nanoscale wires and 1, 2, 3, or 4, etc. n-type nanoscale wires may each be in electrical communication with a cell, and may be arranged in any suitable arrangement, for example, on an alternating basis.

In some cases, the nanoscale wires may be positioned relatively close to each other. For instance, the nanoscale wires may be positioned such that they are separated by a distance of no more than about 200 nm, about 150 nm, or about 100 nm. In some cases, the nanoscale wires may be positioned such that they are substantially parallel to each other. For instance, the nanoscale wires may be positioned such that the nanoscale wires are disposed between first and second electrical connectors (one or both of which electrical connectors may also be substantially parallel), and the cell may be positioned such that it is in contact with at least some of the nanoscale wires.

In one set of embodiments, the nanoscale wires and/or the cells are positioned on the surface of a substrate. Suitable substrates and substrate materials are discussed in more detail below. In some cases, the surface of the substrate may be treated in any fashion that allows binding of cells to occur thereto. For example, the surface may be ionized and/or coated with any of a wide variety of hydrophilic and/or cytophilic materials, for example, materials having exposed carboxylic acid, alcohol, and/or amino groups. In another set of embodiments, the surface of the substrate may be reacted in such a manner as to produce carboxylic acid, alcohol, and/or amino groups on the surface. In some cases, a cell adhesion factor may be used to facilitate adherence of the cells to the substrate, i.e., a biological material that promotes adhesion or binding of cells, for example, materials such as polylysine or other polyamino acids, fibronectin, laminin, vitronectin, albumin, collagen, or peptides or proteins containing RGD sequences. The cell adhesion factor may be deposited on all, or at least a portion of, the substrate.

Another aspect of the invention provides electrical devices including cells, such as neurons, positioned in electrical communication with one or more nanoscale wires, such as previously described. In one set of embodiments, the electrical device is a logic gate, for example, an OR gate or a NOR gate. A NOR gate is a logic gate which outputs 0 if any of the inputs are 1, but outputs 1 if all inputs are 0. The logic gate may comprise more than 2 inputs, i.e., the logic gate is a multi-input logic gate. In some cases, the logic gate may comprise one or more cells (which may each be in electronic communication with each other, for example, if one or more of eth cells are neurons), and a plurality of nanoscale wires positioned in electrical communication with the one or more cells, where one or more of the nanoscale wires act as inputs and one or more nanoscale wires act as outputs. For instance, one nanoscale wire may act as an output (e.g., such that the electrical state of a nanoscale wire is determined in some fashion, using techniques known to those of ordinary skill in the art), while other nanoscale wires are used as inputs to one or more cells (e.g., such that the nanoscale wires are used to electrically stimulate or inhibit the cells, e.g., via polarization, hyperpolarization, depolarization, etc.). In such cases, one (or more) cells may be used as a component of a logic device. Accordingly, in another set of embodiments, computational devices, comprising cells and nanoscale wires (e.g., as logic devices), may be fabricated using the systems and methods described herein.

Certain aspects of the present invention include a nanoscopic wire or other nanostructured material comprising one or more semiconductor and/or metal compounds, for example, for use in any of the above-described embodiments. In some cases, the semiconductors and/or metals may be chemically and/or physically combined, for example, as in a doped nanoscopic wire. The nanoscopic wire may be, for example, a nanorod, a nanowire, a nanowhisker, or a nanotube. The nanoscopic wire may be used in a device, for example, as a semiconductor component, a pathway, etc. The criteria for selection of nanoscale wires and other conductors or semiconductors for use in the invention are based, in some instances, upon whether the nanoscale wire is able to interact with an analyte, or whether the appropriate reaction entity, e.g. a binding partner, can be easily attached to the surface of the nanoscale wire, or the appropriate reaction entity, e.g. a binding partner, is near the surface of the nanoscale wire. Selection of suitable conductors or semiconductors, including nanoscale wires, will be apparent and readily reproducible by those of ordinary skill in the art with the benefit of the present disclosure.

Many nanoscopic wires as used in accordance with the present invention are individual nanoscopic wires. As used herein, “individual nanoscopic wire” means a nanoscopic wire free of contact with another nanoscopic wire (but not excluding contact of a type that may be desired between individual nanoscopic wires, e.g., as in a crossbar array). For example, an “individual” or a “free-standing” article may, at some point in its life, not be attached to another article, for example, with another nanoscopic wire, or the free-standing article may be in solution. This is in contrast to nanotubes produced primarily by laser vaporization techniques that produce materials formed as ropes having diameters of about 2 nm to about 50 nm or more and containing many individual nanotubes. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” article is one that can be (but need not be) removed from the location where it is made, as an individual article, and transported to a different location and combined with different components to make a functional device such as those described herein and those that would be contemplated by those of ordinary skill in the art upon reading this disclosure.

In another set of embodiments, the nanoscopic wire (or other nanostructured material) may include additional materials, such as semiconductor materials, dopants, organic compounds, inorganic compounds, etc. The following are non-limiting examples of materials that may be used as dopants within the nanoscopic wire. The dopant may be an elemental semiconductor, for example, silicon, germanium, tin, selenium, tellurium, boron, diamond, or phosphorous. The dopant may also be a solid solution of various elemental semiconductors. Examples include a mixture of boron and carbon, a mixture of boron and P(BP₆), a mixture of boron and silicon, a mixture of silicon and carbon, a mixture of silicon and germanium, a mixture of silicon and tin, a mixture of germanium and tin, etc. In some embodiments, the dopant may include mixtures of Group IV elements, for example, a mixture of silicon and carbon, or a mixture of silicon and germanium. In other embodiments, the dopant may include mixtures of Group III and Group V elements, for example, BN, BP, BAs, AlN, AlP, AlAs, AlSb, GaN, GaP, GaAs, GaSb, InN, InP, InAs, or InSb. Mixtures of these combinations may also be used, for example, a mixture of BN/BP/BAs, or BN/AlP. In other embodiments, the dopants may include mixtures of Group III and Group V elements. For example, the mixtures may include AlGaN, GaPAs, InPAs, GaInN, AlGaInN, GaInAsP, or the like. In other embodiments, the dopants may also include mixtures of Group II and Group VI elements. For example, the dopant may include mixtures of ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also be possible, for example, ZnCd Se, or ZnSSe or the like. Additionally, mixtures of different groups of semiconductors may also be possible, for example, combinations of Group II-Group VI and Group III-Group V elements, such as (GaAs)ₓ(ZnS)₁₋ₓ. Other non-limiting examples of dopants may include mixtures of Group IV and Group VI elements, for example GeS, GeSe, GeTe, SnS, SnSe, SnTe, PbO, PbS, PbSe, PbTe, etc. Other dopant mixtures may include mixtures of Group I elements and Group VII elements, such as CuF, CuCl, CuBr, CuI, AgF, AgCl, AgBr, AgI, or the like. Other dopant mixtures may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ or the like.

As a non-limiting example, a p-type dopant may be selected from Group III, and an n-type dopant may be selected from Group V. For instance, a p-type dopant may include at least one of B, Al and In, and an n-type dopant may include at least one of P, As and Sb. For Group III-Group V mixtures, a p-type dopant may be selected from Group II, including one or more of Mg, Zn, Cd and Hg, or Group IV, including one or more of C and Si. An n-type dopant may be selected from at least one of Si, Ge, Sn, S, Se and Te. It will be understood that the invention is not limited to these dopants, but may include other elements, alloys, or mixtures as well.

As used herein, the term “Group,” with reference to the Periodic Table, is given its usual definition as understood by one of ordinary skill in the art. For instance, the Group II elements include Mg and Ca, as well as the Group II transition elements, such as Zn, Cd, and Hg. Similarly, the Group III elements include B, Al, Ga, In and Tl; the Group IV elements include C, Si, Ge, Sn, and Pb; the Group V elements include N, P, As, Sb and Bi; and the Group VI elements include O, S, Se, Te and Po. Combinations involving more than one element from each Group are also possible. For example, a Group II-VI material may include at least one element from Group II and at least one element from Group VI, e.g., ZnS, ZnSe, ZnSSe, ZnCdS, CdS, or CdSe. Similarly, a Group III-V material may include at least one element from Group III and at least one element from Group V, for example GaAs, GaP, GaAsP, InAs, InP, AlGaAs, or InAsP. Other dopants may also be included with these materials and combinations thereof, for example, transition metals such as Fe, Co, Te, Au, and the like. The nanoscale wire of the present invention may further include, in some cases, any organic or inorganic molecules. In some cases, the organic or inorganic molecules are polarizable and/or have multiple charge states.

In some embodiments, at least a portion of a nanoscopic wire may be a bulk-doped semiconductor. As used herein, a “bulk-doped” article (e. g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles such as carbon nanotubes are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. In particular, in some embodiments, a bulk-doped semiconductor may comprise two or more bulk-doped regions. Thus, as used herein to describe nanoscopic wires, “doped” refers to bulk-doped nanoscopic wires, and, accordingly, a “doped nanoscopic (or nanoscale) wire” is a bulk-doped nanoscopic wire. “Heavily doped” and “lightly doped” are terms the meanings of which are understood by those of ordinary skill in the art.

In one set of embodiments, the invention includes a nanoscale wire (or other nanostructured material) that is a single crystal. As used herein, a “single crystal” item (e.g., a semiconductor) is an item that has covalent bonding, ionic bonding, or a combination thereof throughout the item. Such a single-crystal item may include defects in the crystal, but is to be distinguished from an item that includes one or more crystals, not ionically or covalently bonded, but merely in close proximity to one another.

In yet another set of embodiments, the nanoscale wire (or other nanostructured material) may comprise two or more regions having different compositions. Each region of the nanoscale wire may have any shape or dimension, and these can be the same or different between regions. For example, a region may have a smallest dimension of less than 1 micron, less than 100 nm, less than 10 nm, or less than 1 nm. In some cases, one or more regions may be a single monolayer of atoms (i.e., “delta-doping”). In certain cases, the region may be less than a single monolayer thick (for example, if some of the atoms within the monolayer are absent).

The two or more regions may be longitudinally arranged relative to each other, and/or radially arranged (e.g., as in a core/shell arrangement) within the nanoscale wire. As one example, the nanoscale wire may have multiple regions of semiconductor materials arranged longitudinally. In another example, a nanoscale wire may have two regions having different compositions arranged longitudinally, surrounded by a third region or several regions, each having a composition different from that of the other regions. As a specific example, the regions may be arranged in a layered structure within the nanoscale wire, and one or more of the regions may be delta-doped or at least partially delta-doped. As another example, the nanoscale wire may have a series of regions positioned both longitudinally and radially relative to each other. The arrangement can include a core that differs in composition along its length (changes in composition or concentration longitudinally), while the lateral (radial) dimensions of the core do, or do not, change over the portion of the length differing in composition. The shell portions can be adjacent each other (contacting each other, or defining a change in composition or concentration of a unitary shell structure longitudinally), or can be separated from each other by, for example, air, an insulator, a fluid, or an auxiliary, non-nanoscale wire component. The shell portions can be positioned directly on the core, or can be separated from the core by one or more intermediate shells portions that can themselves be constant in composition longitudinally, or varying in composition longitudinally, i.e., the invention allows the provision of any combination of a nanowire core and any number of radially-positioned shells (e.g., concentric shells), where the core and/or any shells can vary in composition and/or concentration longitudinally, any shell sections can be spaced from any other shell sections longitudinally, and different numbers of shells can be provided at different locations longitudinally along the structure.

In still another set of embodiments, a nanoscale wire may be positioned proximate the surface of a substrate, i.e., the nanoscale wire may be positioned within about 50 nm, about 25 nm, about 10 nm, or about 5 nm of the substrate. In some cases, the proximate nanoscale wire may contact at least a portion of the substrate. In one embodiment, the substrate comprises a semiconductor and/or a metal. Non-limiting examples include Si, Ge, GaAs, etc. Other suitable semiconductors and/or metals are described above with reference to nanoscale wires. In certain embodiments, the substrate may comprise a nonmetal/nonsemiconductor material, for example, a glass, a plastic or a polymer, a gel, a thin film, etc. Non-limiting examples of suitable polymers that may form or be included in the substrate include polyethylene, polypropylene, poly(ethylene terephthalate), polydimethylsiloxane, or the like.

In certain aspects, the present invention provides a method of preparing a nanostructure. In certain embodiments, the present invention involves controlling and altering the doping of semiconductors in a nanoscale wire. In some cases, the nanoscale wires (or other nanostructure) may be produced using techniques that allow for direct and controlled growth of the nanoscale wires. In some cases, the nanoscale wire may be doped during growth of the nanoscale wire. Doping the nanoscale wire during growth may result in the property that the doped nanoscale wire is bulk-doped. Furthermore, such doped nanoscale wires may be controllably doped, such that a concentration of a dopant within the doped nanoscale wire can be controlled and therefore reproduced consistently.

Certain arrangements may utilize metal-catalyzed CVD techniques (“chemical vapor deposition”) to synthesize individual nanoscale wires. CVD synthetic procedures useful for preparing individual wires directly on surfaces and in bulk form are generally known, and can readily be carried out by those of ordinary skill in the art. Nanoscopic wires may also be grown through laser catalytic growth. With the same basic principles as LCG, if uniform diameter nanoclusters (less than 10% to 20% variation depending on how uniform the nanoclusters are) are used as the catalytic cluster, nanoscale wires with uniform size (diameter) distribution can be produced, where the diameter of the wires is determined by the size of the catalytic clusters. By controlling growth time, nanoscale wires with different lengths can be grown.

One technique that may be used to grow nanoscale wires is catalytic chemical vapor deposition (“C-CVD”). In C-CVD, reactant molecules are formed from the vapor phase. Nanoscale wires may be doped by introducing the doping element into the vapor phase reactant (e.g. diborane and phosphane). The doping concentration may be controlled by controlling the relative amount of the doping compound introduced in the composite target. The final doping concentration or ratios are not necessarily the same as the vapor-phase concentration or ratios. By controlling growth conditions, such as temperature, pressure or the like, nanoscale wires having the same doping concentration may be produced.

Another technique for direct fabrication of nanoscale wire junctions during synthesis is referred to as laser catalytic growth (“LCG”). In LCG, dopants are controllably introduced during vapor phase growth of nanoscale wires. Laser vaporization of a composite target composed of a desired material (e.g. silicon or indium phosphide) and a catalytic material (e.g. a nanoparticle catalyst) may create a hot, dense vapor. The vapor may condense into liquid nanoclusters through collision with a buffer gas. Growth may begin when the liquid nanoclusters become supersaturated with the desired phase and can continue as long as reactant is available. Growth may terminate when the nanoscale wire passes out of the hot reaction zone and/or when the temperature is decreased. The nanoscale wire may be further subjected to different semiconductor reagents during growth.

Other techniques to produce nanoscale semiconductors such as nanoscale wires are also contemplated. For example, nanoscale wires of any of a variety of materials may be grown directly from vapor phase through a vapor-solid process. Also, nanoscale wires may also be produced by deposition on the edge of surface steps, or other types of patterned surfaces. Further, nanoscale wires may be grown by vapor deposition in or on any generally elongated template. The porous membrane may be porous silicon, anodic alumina, a diblock copolymer, or any other similar structure. The natural fiber may be DNA molecules, protein molecules carbon nanotubes, any other elongated structures. For all the above described techniques, the source materials may be a solution or a vapor. In some cases, while in solution phase, the template may also include be column micelles formed by surfactant.

In some cases, the nanoscale wire may be doped after formation. In one technique, a nanoscale wire having a substantially homogeneous composition is first synthesized, then is doped post-synthetically with various dopants. Such doping may occur throughout the entire nanoscale wire, or in one or more portions of the nanoscale wire, for example, in a wire having multiple regions differing in composition.

In one set of embodiments, the method involves allowing a first material to diffuse into at least part of a second material, optionally creating a new compound. For example, the first and second materials may each be metals or semiconductors, one material may be a metal and the other material may be a semiconductor, etc. In one set of embodiments, a semiconductor may be annealed to a metal. For example, a portion of the semiconductor and/or a portion of the metal may be heated such that at least some metal atoms are able to diffuse into the semiconductor, or vice versa. In one embodiment, a metal electrode (e.g., a nickel, gold, copper, silver, chromium electrode, etc.), may be positioned in physical contact with a semiconductor nanoscopic wire, and then annealed such that at least a portion of the semiconductor diffuses into at least a portion of the metal, optionally forming a metal-semiconductor compound, e.g., as disclosed in International Patent Application No. PCT/US2005/004459, filed Feb. 14, 2005, entitled “Nanostructures Containing Metal-Semiconductor Compounds,” by Lieber, et al., incorporated herein by reference. For example, the semiconductor may be annealed with the metal at a temperature of about 300° C., about 350° C., about 400° C., about 450° C., about 500° C., about 550° C., or about 600° C. for a period of time of at least about 30 minutes, at least about 1 hour, at least about 2 hours, at least about 4 hours, at least about 6 hours etc. Such annealing may allow, for example, lower contact resistances or impedances between the metal and the semiconductor.

In some cases, the metal may be passivated, e.g., as described herein. For example, the metal, or at least a portion of the metal, may be exposed to one or more passivating agents, for example, Si₃N₄. Insulation of the metal by the passivating agent may be used to form a layer covering the surface of the metal, for example, to prevent reaction or nonspecific binding between an analyte and the metal. For instance, a metal electrode may be in electrical communication with a semiconductor comprising one or more immobilized reaction entities, and the metal electrode may be passivated to prevent a reaction or nonspecific binding between the metal and the reaction entity, and/or to reduce or prevent leakage current from the metal. In some cases, the passivation may be conducted at a relatively high temperature, for example, within a plasma CVD chamber.

One aspect of the invention provides for the assembly, or controlled placement, of nanoscale wires on a surface. Any substrate may be used for nanoscale wire placement, for example, a substrate comprising a semiconductor, a substrate comprising a metal, a substrate comprising a glass, a substrate comprising a polymer, a substrate comprising a gel, a substrate that is a thin film, a substantially transparent substrate, a non-planar substrate, a flexible substrate, a curved substrate, etc. In some cases, assembly can be carried out by aligning nanoscale wires using an electrical field. In other cases, assembly can be performed using an arrangement involving positioning a fluid flow directing apparatus to direct fluid containing suspended nanoscale wires toward and in the direction of alignment with locations at which nanoscale wires are desirably positioned.

In certain cases, a nanoscale wire (or other nanostructure) is formed on the surface of a substrate, and/or is defined by a feature on a substrate. In one example, a nanostructure, such as a nanoscale wire, is formed as follows. A substrate is imprinted using a stamp or other applicator to define a pattern, such as a nanoscale wire or other nanoscale structure. After removal of the stamp or other applicator, at least a portion of the imprintable layer is removed, for example, through etching processes such as reactive ion etching (RIE), or other known techniques. In some cases, enough imprintable material may be removed from the substrate so as to expose portions of the substrate free of the imprintable material. A metal or other materials may then be deposited onto at least a portion of the substrate, for example, gold, copper, silver, chromium, etc. In some cases, a “lift-off” step may then be performed, where at least a portion of the imprintable material is removed from the substrate. Metal or other material deposited onto the imprintable material may be removed along with the removal of the imprintable material, for example, to form one or more nanoscale wires. Structures deposited on the surface may be connected to one or more electrodes in some cases. The substrate may be any suitable substrate that can support an imprintable layer, for example, comprising a semiconductor, a metal, a glass, a polymer, a gel, etc. In some cases, the substrate may be a thin film, substantially transparent, non-planar, flexible, and/or curved, etc.

In certain cases, an array of nanoscale wires may be produced by providing a surface having a plurality of substantially aligned nanoscale wires, and removing, from the surface, a portion of one or more of the plurality of nanoscale wires. The remaining nanoscale wires on the surface may then be connected to one or more electrodes. In certain cases, the nanoscopic wires are arranged such that they are in contact with each other; in other instances, however, the aligned nanoscopic wires may be at a pitch such that they are substantially not in physical contact.

In certain cases, nanoscale wires are positioned proximate a surface using flow techniques, i.e., techniques where one or more nanoscale wires may be carried by a fluid to a substrate. Nanoscale wires (or any other elongated structures) can be aligned by inducing a flow of a nanoscale wire solution on surface, where the flow can include channel flow or flow by any other suitable technique. Nanoscale wire arrays with controlled position and periodicity can be produced by patterning a surface of a substrate and/or conditioning the surface of the nanoscale wires with different functionalities, where the position and periodicity control may be achieved by designing specific complementary forces between the patterned surface and the nanoscale wires. Nanoscale wires can also be assembled using a Langmuir-Blodgett (LB) trough. Nanoscale wires may first be surface-conditioned and dispersed to the surface of a liquid phase to form a Langmuir-Blodgett film. In some cases, the liquid may include a surfactant, which can, in some cases, reduce aggregation of the nanoscale wires and/or reduce the ability of the nanoscale wires to interact with each other. The nanoscale wires can be aligned into different patterns (such as parallel arrays or fibers) by compressing the surface or reducing the surface area of the surface.

Another arrangement involves forming surfaces on a substrate including regions that selectively attract nanoscale wires surrounded by regions that do not selectively attract them. Surfaces can be patterned using known techniques such as electron-beam patterning, “soft-lithography” such as that described in International Patent Application Serial No. PCT/US96/03073, entitled “Microcontact Printing on Surfaces and Derivative Articles,” filed Mar. 1, 1996, published as Publication No. WO 96/29629 on Jul. 26, 1996; or U.S. Pat. No. 5,512,131, entitled “Formation of Microstamped Patterns on Surfaces and Derivative Articles,” issued Apr. 30, 1996, each of which is incorporated herein by reference. Additional techniques are described in U.S. Patent Application Ser. No. 60/142,216, entitled “Molecular Wire-Based Devices and Methods of Their Manufacture,” filed Jul. 2, 1999, incorporated herein by reference. Fluid flow channels can be created at a size scale advantageous for placement of nanoscale wires on surfaces using a variety of techniques such as those described in International Patent Application Serial No. PCT/US97/04005, entitled “Method of Forming Articles and Patterning Surfaces via Capillary Micromolding,” filed Mar. 14,1997, published as Publication No. WO 97/33737 on Sep. 18, 1997, and incorporated herein by reference. Other techniques include those described in U.S. Pat. No. 6,645,432, entitled “Microfluidic Systems Including Three-dimensionally Arrayed Channel Networks,” issued Nov. 11, 2003, incorporated herein by reference.

Chemically patterned surfaces other than SAM-derivatized surfaces can be used, and many techniques for chemically patterning surfaces are known. Another example of a chemically patterned surface may be a micro-phase separated block copolymer structure. These structures may provide a stack of dense lamellar phases, where a cut through these phases reveals a series of “lanes” wherein each lane represents a single layer. The assembly of nanoscale wires onto substrate and electrodes can also be assisted using bimolecular recognition in some cases. For example, one biological binding partner may be immobilized onto the nanoscale wire surface and the other one onto a substrate or an electrode using physical adsorption or covalently linking. An example technique which may be used to direct the assembly of a nanoscopic wires on a substrate is by using “SAMs,” or self-assembled monolayers. Any of a variety of substrates and SAM-forming material can be used along with microcontact printing techniques, such as those described in International Patent Application Serial No. PCT/US96/03073, entitled “Microcontact Printing on Surfaces and Derivative Articles,” filed Mar. 1, 1996, published as Publication No. WO 96/29629 on Jul. 26, 1996, incorporated herein by reference in its entirety.

In some cases, the nanoscale wire arrays may also be transferred to another substrate, e.g., by using stamping techniques. In certain instances, nanoscale wires may be assembled using complementary interaction, i.e., where one or more complementary chemical, biological, electrostatic, magnetic or optical interactions are used to position one or more nanoscale wires on a substrate. In certain cases, physical patterns may be used to position nanoscale wires proximate a surface. For example, nanoscale wires may be positioned on a substrate. using physical patterns, for instance, aligning the nanoscale wires using corner of the surface steps or along trenches on the substrate.

In one aspect, the present invention provides any of the above-mentioned devices packaged in kits, optionally including instructions for use of the devices. As used herein, “instructions” can define a component of instructional utility (e.g., directions, guides, warnings, labels, notes, FAQs (“frequently asked questions”), etc., and typically involve written instructions on or associated with packaging of the invention. Instructions can also include instructional communications in any form (e.g., oral, electronic, digital, optical, visual, etc.), provided in any manner such that a user will clearly recognize that the instructions are to be associated with the device, e.g., as discussed herein. Additionally, the kit may include other components depending on the specific application, for example, containers, adapters, syringes, needles, replacement parts, etc. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, replacing, or the like that can be associated with the methods and compositions of the invention, e.g., as discussed herein. Promoting may also include, in some cases, seeking approval from a government agency to sell a composition of the invention for medicinal purposes. Methods of promotion can be performed by any party including, but not limited to, businesses (public or private), contractual or sub-contractual agencies, educational institutions such as colleges and universities, research institutions, hospitals or other clinical institutions, governmental agencies, etc. Promotional activities may include instructions or communications of any form (e.g., written, oral, and/or electronic communications, such as, but not limited to, e-mail, telephonic, facsimile, Internet, Web-based, etc.) that are clearly associated with the invention.

The following definitions will aid in the understanding of the invention. Certain devices of the invention may include wires or other components of scale commensurate with nanometer-scale wires, which includes nanotubes and nanowires. In some embodiments, however, the invention comprises articles that may be greater than nanometer size (e. g., micrometer-sized). As used herein, “nanoscopic-scale,” “nanoscopic,” “nanometer-scale,” “nanoscale,” the “nano-” prefix (for example, as in “nanostructured”), and the like generally refers to elements or articles having widths or diameters of less than about 1 micron, and less than about 100 nm in some cases. In all embodiments, specified widths can be a smallest width (i.e. a width as specified where, at that location, the article can have a larger width in a different dimension), or a largest width (i.e. where, at that location, the article has a width that is no wider than as specified, but can have a length that is greater).

As used herein, a “wire” generally refers to any material having a conductivity of or of similar magnitude to any semiconductor or any metal, and in some embodiments may be used to connect two electronic components such that they are in electronic communication with each other. For example, the terms “electrically conductive” or a “conductor” or an “electrical conductor” when used with reference to a “conducting” wire or a nanoscale wire, refers to the ability of that wire to pass charge. Typically, an electrically conductive nanoscale wire will have a resistivity comparable to that of metal or semiconductor materials, and in some cases, the electrically conductive nanoscale wire may have lower resistivities, for example, resistivities of less than about 100 microOhm cm (μΩ cm). In some cases, the electrically conductive nanoscale wire will have a resistivity lower than about 10⁻³ ohm meters, lower than about 10⁻⁴ ohm meters, or lower than about 10⁻⁶ ohm meters or 10⁻⁷ ohm meters.

A “semiconductor,” as used herein, is given its ordinary meaning in the art, i.e., an element having semiconductive or semi-metallic properties (i.e., between metallic and non-metallic properties). An example of a semiconductor is silicon. Other non-limiting examples include gallium, germanium, diamond (carbon), tin, selenium, tellurium, boron, or phosphorous.

A “nanoscopic wire” (also known herein as a “nanoscopic-scale wire” or “nanoscale wire”) generally is a wire, that at any point along its length, has at least one cross-sectional dimension and, in some embodiments, two orthogonal cross-sectional dimensions less than 1 micron, less than about 500 nm, less than about 200 nm, less than about 150 nm, less than about 100 nm, less than about 70, less than about 50 nm, less than about 20 nm, less than about 10 nm, or less than about 5 nm. In other embodiments, the cross-sectional dimension can be less than 2 nm or 1 nm. In one set of embodiments, the nanoscale wire has at least one cross-sectional dimension ranging from 0.5 nm to 100 nm or 200 nm. In some cases, the nanoscale wire is electrically conductive. Where nanoscale wires are described having, for example, a core and an outer region, the above dimensions generally relate to those of the core. The cross-section of a nanoscopic wire may be of any arbitrary shape, including, but not limited to, circular, square, rectangular, annular, polygonal, or elliptical, and may be a regular or an irregular shape. The nanoscale wire may be solid or hollow. A non-limiting list of examples of materials from which nanoscale wires of the invention can be made appears below. Any nanoscale wire can be used in any of the embodiments described herein, including carbon nanotubes, molecular wires (i.e., wires formed of a single molecule), nanorods, nanowires, nanowhiskers, organic or inorganic conductive or semiconducting polymers, and the like, unless otherwise specified. Other conductive or semiconducting elements that may not be molecular wires, but are of various small nanoscopic-scale dimensions, can also be used in some instances, e.g. inorganic structures such as main group and metal atom-based wire-like silicon, transition metal-containing wires, gallium arsenide, gallium nitride, indium phosphide, germanium, cadmium selenide, etc. A wide variety of these and other nanoscale wires can be grown on and/or applied to surfaces in patterns useful for electronic devices in a manner similar to techniques described herein involving the specific nanoscale wires used as examples, without undue experimentation. The nanoscale wires, in some cases, may be formed having dimensions of at least about 1 micron, at least about 3 microns, at least about 5 microns, or at least about 10 microns or about 20 microns in length, and can be less than about 100 nm, less than about 80 nm, less than about 60 nm, less than about 40 nm, less than about 20 nm, less than about 10 nm, or less than about 5 nm in thickness (height and width). The nanoscale wires may have an aspect ratio (length to thickness) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

A “nanowire” (e. g. comprising silicon and/or another semiconductor material) is a nanoscopic wire that is typically a solid wire, and may be elongated in some cases. Preferably, a nanowire (which is abbreviated herein as “NW”) is an elongated semiconductor, i.e., a nanoscale semiconductor. A “non-nanotube nanowire” is any nanowire that is not a nanotube. In one set of embodiments of the invention, a non-nanotube nanowire having an unmodified surface (not including an auxiliary reaction entity not inherent in the nanotube in the environment in which it is positioned) is used in any arrangement of the invention described herein in which a nanowire or nanotube can be used.

As used herein, a “nanotube” (e.g. a carbon nanotube) is a nanoscopic wire that is hollow, or that has a hollowed-out core, including those nanotubes known to those of ordinary skill in the art. “Nanotube” is abbreviated herein as “NT.” Nanotubes are used as one example of small wires for use in the invention and, in certain embodiments, devices of the invention include wires of scale commensurate with nanotubes. Examples of nanotubes that may be used in the present invention include, but are not limited to, single-walled nanotubes (SWNTs). Structurally, SWNTs are formed of a single graphene sheet rolled into a seamless tube. Depending on the diameter and helicity, SWNTs can behave as one-dimensional metals and/or semiconductors. SWNTs. Methods of manufacture of nanotubes, including SWNTs, and characterization are known. Methods of selective functionalization on the ends and/or sides of nanotubes also are known, and the present invention makes use of these capabilities for molecular electronics in certain embodiments. Multi-walled nanotubes are well known, and can be used as well.

As used herein, an “elongated” article (e.g. a semiconductor or a section thereof) is an article for which, at any point along the longitudinal axis of the article, the ratio of the length of the article to the largest width at that point is greater than 2:1.

A “width” of an article, as used herein, is the distance of a straight line from a point on a perimeter of the article, through the center of the article, to another point on the perimeter of the article. As used herein, a “width” or a “cross-sectional dimension” at a point along a longitudinal axis of an article is the distance along a straight line that passes through the center of a cross-section of the article at that point and connects two points on the perimeter of the cross-section. The “cross-section” at a point along the longitudinal axis of an article is a plane at that point that crosses the article and is orthogonal to the longitudinal axis of the article. The “longitudinal axis” of an article is the axis along the largest dimension of the article. Similarly, a “longitudinal section” of an article is a portion of the article along the longitudinal axis of the article that can have any length greater than zero and less than or equal to the length of the article. Additionally, the “length” of an elongated article is a distance along the longitudinal axis from end to end of the article.

As used herein, a “cylindrical” article is an article having an exterior shaped like a cylinder, but does not define or reflect any properties regarding the interior of the article. In other words, a cylindrical article may have a solid interior, may have a hollowed-out interior, etc. Generally, a cross-section of a cylindrical article appears to be circular or approximately circular, but other cross-sectional shapes are also possible, such as a hexagonal shape. The cross-section may have any arbitrary shape, including, but not limited to, square, rectangular, or elliptical. Regular and irregular shapes are also included.

As used herein, an “array” of articles (e.g., nanoscopic wires) comprises a plurality of the articles, for example, a series of aligned nanoscale wires, which may or may not be in contact with each other. As used herein, a “crossed array” or a “crossbar array” is an array where at least one of the articles contacts either another of the articles or a signal node (e.g., an electrode). “Determine,” as used herein, generally refers to the analysis of a state or condition, for example, quantitatively or qualitatively. For example, a species, or an electrical state of a system may be determined. “Determining” may also refer to the analysis of an interaction between two or more species, for example, quantitatively or qualitatively, and/or by detecting the presence or absence of the interaction, e.g. determination of the binding between two species. As an example, an analyte may cause a determinable change in an electrical property of a nanoscale wire (e.g., electrical conductivity, resistivity, impedance, etc.), a change in an optical property of the nanoscale wire, etc. Examples of determination techniques include, but are not limited to, conductance measurement, current measurement, voltage measurement, resistance measurement, piezoelectric measurement, electrochemical measurement, electromagnetic measurement, photodetection, mechanical measurement, acoustic measurement, gravimetric measurement, and the like. “Determining” also means detecting or quantifying interaction between species.

A “fluid,” as used herein, generally refers to a substance that tends to flow and to conform to the outline of its container. Typically, fluids are materials that are unable to withstand a static shear stress. When a shear stress is applied to a fluid, it experiences a continuing and permanent distortion. Typical fluids include liquids and gases, but may also include free-flowing solid particles, viscoelastic fluids, and the like.

As used herein, a component that is “immobilized relative to” another component either is fastened to the other component or is indirectly fastened to the other component, e.g., by being fastened to a third component to which the other component also is fastened. For example, a first entity is immobilized relative to a second entity if a species fastened to the surface of the first entity attaches to an entity, and a species on the surface of the second entity attaches to the same entity, where the entity can be a single entity, a complex entity of multiple species, another particle, etc. In certain embodiments, a component that is immobilized relative to another component is immobilized using bonds that are stable, for example, in solution or suspension. In some embodiments, non-specific binding of a component to another component, where the components may easily separate due to solvent or thermal effects, is not preferred.

As used herein, “fastened to or adapted to be fastened to,” as used in the context of a species relative to another species or a species relative to a surface of an article (such as a nanoscale wire), or to a surface of an article relative to another surface, means that the species and/or surfaces are chemically or biochemically linked to or adapted to be linked to, respectively, each other via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a nanoscale wire, a binding species specifically biologically coupled to an antibody which is bound to a protein such as protein A, which is attached to a nanoscale wire, a binding species that forms a part of a molecule, which in turn is specifically biologically bound to a binding partner covalently fastened to a surface of a nanoscale wire, etc. A species also is adapted to be fastened to a surface if a surface carries a particular nucleotide sequence, and the species includes a complementary nucleotide sequence.

“Specifically fastened” or “adapted to be specifically fastened” means a species is chemically or biochemically linked to or adapted to be linked to, respectively, another specimen or to a surface as described above with respect to the definition of “fastened to or adapted to be fastened,” but excluding essentially all non-specific binding. “Covalently fastened” means fastened via essentially nothing other than one or more covalent bonds.

The term “binding” refers to the interaction between a corresponding pair of molecules or surfaces that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones and the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc.

The term “binding partner” refers to a molecule that can undergo binding with a particular molecule. Biological binding partners are examples. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. For example, Protein A is usually regarded as a “non-specific” or semi-specific binder. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include nucleic acids that specifically bind (hybridize) to their complement, antibodies specifically bind to their antigen, binding pairs such as those described above, and the like. The binding may be by one or more of a variety of mechanisms including, but not limited to ionic interactions, and/or covalent interactions, and/or hydrophobic interactions, and/or van der Waals interactions, etc.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

As used herein, terms such as “polynucleotide” or “oligonucleotide” or grammatical equivalents generally refer to a polymer of at least two nucleotide bases covalently linked together, which may include, for example, but not limited to, natural nucleosides (e.g., adenosine, thymidine, guanosine, cytidine, uridine, deoxyadenosine, deoxythymidine, deoxyguanosine and deoxycytidine), nucleoside analogs (e.g., 2-aminoadenosine, 2-thiothymidine, inosine, pyrrolopyrimidine, 3-methyladenosine, C5-bromouridine, C5-fluorouridine, C5-iodouridine, C5-propynyluridine, C5-propynylcytidine, C5-methylcytidine, 7-deazaadenosine, 7-deazaguanosine, 8-oxoadenosine, 8-oxoguanosine, O6-methylguanosine, 2-thiocytidine, 2-aminopurine, 2-amino-6-chloropurine, 2,6-diaminopurine, hypoxanthine), chemically or biologically modified bases (e.g., methylated bases), intercalated bases, modified sugars (2′-fluororibose, arabinose, or hexose), modified phosphate moieties (e.g., phosphorothioates or 5′-N-phosphoramidite linkages), and/or other naturally and non-naturally occurring bases substitutable into the polymer, including substituted and unsubstituted aromatic moieties. Other suitable base and/or polymer modifications are well-known to those of skill in the art. Typically, an “oligonucleotide” is a polymer having 20 bases or less, and a “polynucleotide” is a polymer having at least 20 bases. Those of ordinary skill in the art will recognize that these terms are not precisely defined in terms of the number of bases present within the polymer strand.

A “nucleic acid,” as used herein, is given its ordinary meaning as used in the art. Nucleic acids can be single-stranded or double stranded, and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1 970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp. 169-176). Several nucleic acid analogs are described in Rawls, Chemical & Engineering News, Jun. 2, 1997 page 35. These modifications of the ribose-phosphate backbone may be done to facilitate the addition of additional moieties such as labels, or to increase the stability and half-life of such molecules in physiological environments.

As used herein, an “antibody” refers to a protein or glycoprotein including one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immnunoglobulins or as a number of well characterized fragments produced by digestion with various peptidases. Thus, for example, pepsin digests an antibody below (i.e. toward the Fc domain) the disulfide linkages in the hinge region to produce F(ab)'2, a dimer of Fab which itself is a light chain joined to VH-CHI by a disulfide bond. The F(ab)'2 may be reduced under mild conditions to break the disulfide linkage in the hinge region thereby converting the (Fab')2 dimer into an Fab' monomer. The Fab' monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g., single chain Fv (scFv) antibodies in which a variable heavy and a variable light chain are joined together (directly or through a peptide linker) to form a continuous polypeptide.

The term “quantum dot” is known to those of ordinary skill in the art, and generally refers to semiconductor or metal nanoparticles that absorb light and quickly re-emit light in a different color depending on the size of the dot. For example, a 2 nanometer quantum dot emits green light, while a 5 nanometer quantum dot emits red light. Cadmium selenide quantum dot nanocrystals are available from Quantum Dot Corporation of Hayward, Calif.

The following documents are each incorporated herein by reference: U.S. patent application Ser. No. 09/935,776, filed Aug. 22, 2001, entitled “Doped Elongated Semiconductors, Growing Such Semiconductors, Devices Including Such Semiconductors, and Fabricating Such Devices,” by Lieber, et al., published as U.S. Patent Application Publication No. 2002/0130311 on Sep. 19, 2002; U.S. patent application Ser. No. 10/020,004, filed Dec. 11, 2001, entitled “Nanosensors,” by Lieber, et al., published as U.S. Patent Application Publication No. 2002/0117659 on Aug. 29, 2002; U.S. patent application Ser. No. 10/196,337, filed Jul. 16, 2002, entitled “Nanoscale Wires and Related Devices,” by Lieber, et al., published as U.S. Patent Application Publication No. 2003/0089899 on May 15, 2003; U.S. patent application Ser. No. 10/995,075, filed Nov. 22, 2004, entitled “Nanoscale Arrays, Robust Nanostructures, and Related Devices,” by Whang, et al., published as U.S. Patent Application Publication No. 2005/0253137 on Nov. 17, 2005; U.S. Provisional Patent Application Ser. No. 60/551,634, filed Mar. 8, 2004, entitled “Robust Nanostructures,” by McAlpine, et al.; International Patent Application No. PCT/US2005/004459, filed Feb. 14, 2005, entitled “Nanostructures Containing Metal-Semiconductor Compounds,” by Lieber, et al., published as WO 2005/093831 on Oct. 6, 2005; International Patent Application No. PCT/US2005/020974, filed Jun. 15, 2005, entitled “Nanosensors,” by Wang, et al.; U.S. patent application Ser. No. 11/137,784, filed May 25, 2005, entitled “Nanoscale Sensors,” by Lieber, et al.; an International Patent Application filed Sep. 21, 2005, entitled “Nanowire Heterostructures,” by Lu, et al.; U.S. Provisional Patent Application Ser. No. 60/707,136, filed Aug. 9, 2005, entitled “Nanoscale Sensors,” by Lieber, et al.; and U.S. Provisional Patent Application Ser. No. 60/783,203, filed Mar. 15, 2006, entitled “Nanobioelectronics,” by Patolsky, et al.

The following examples are intended to illustrate certain embodiments of the present invention, but do not exemplify the full scope of the invention.

### EXAMPLE 1

The interface between nanoscale semiconductors and biological systems represents a powerful means for molecular-scale communication between these two distinct yet complementary components of information processing systems. This example illustrates the assembly and electrical properties of nanowire-based device arrays integrated with mammalian neurons. Discrete hybrid structures enable neuronal recording and stimulation at the axon, dendrite, or soma with high sensitivity and spatial resolution. Aligned arrays of these electronic nanostructures are used to measure the speed and shape evolution of action potentials as well as to interact with a single cell as multiple inputs and outputs. Additionally, we have demonstrated the assembly of hybrid n- and p-type structures enabling the generation of bipolar signals that could form the basis of logic gates and other integrated neuron-based computing structures. The flexible assembly of arrays of these structures creating tens of inputs or outputs to a single cell could prove useful for fundamental neurophysiological studies, real-time cellular interaction with chemical species, and the creation of hybrid cell/semiconductor computational networks.

### EXAMPLE 2

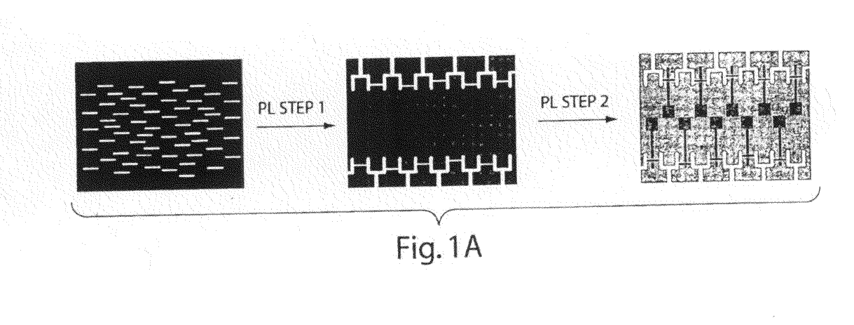

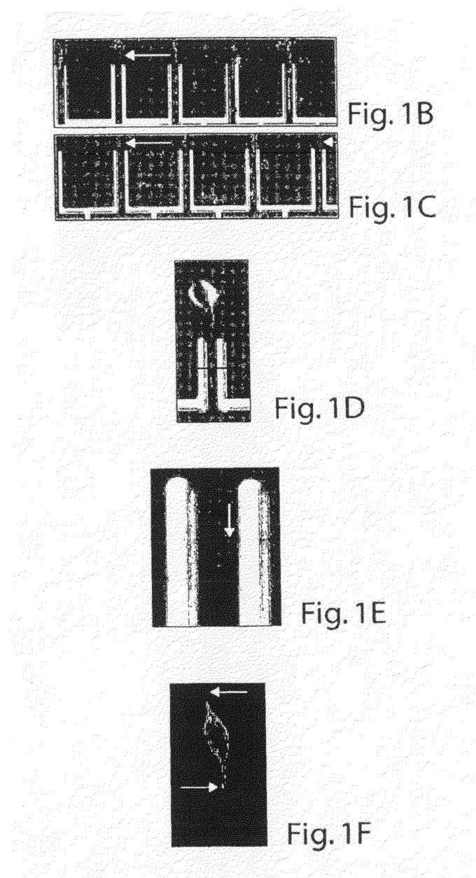

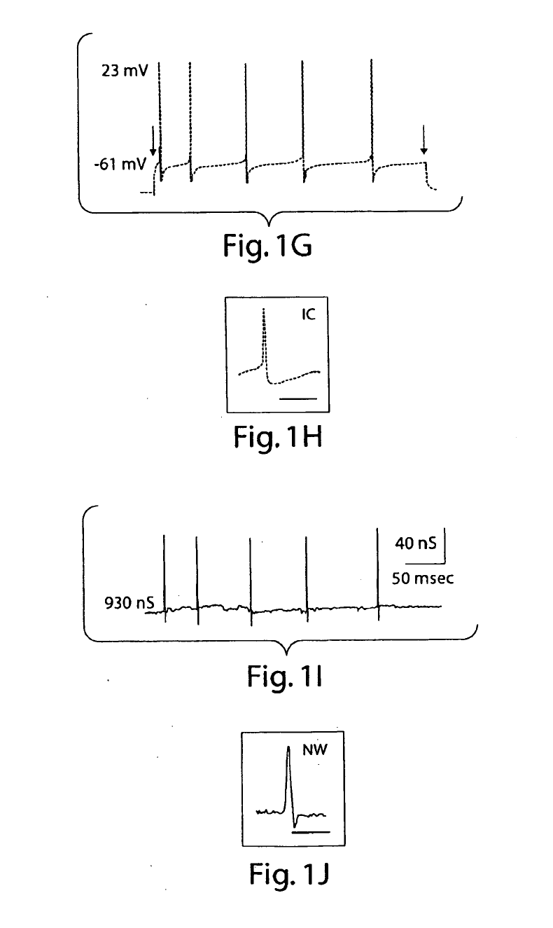

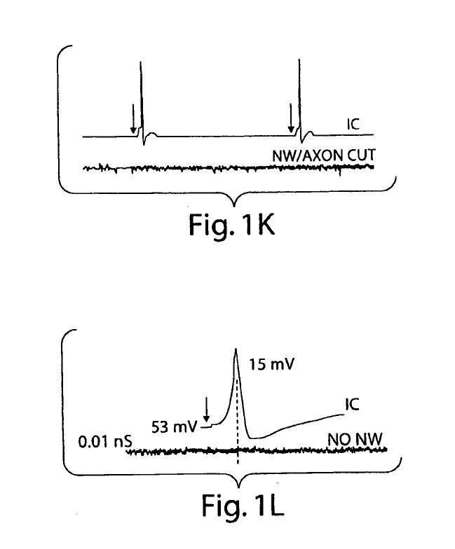

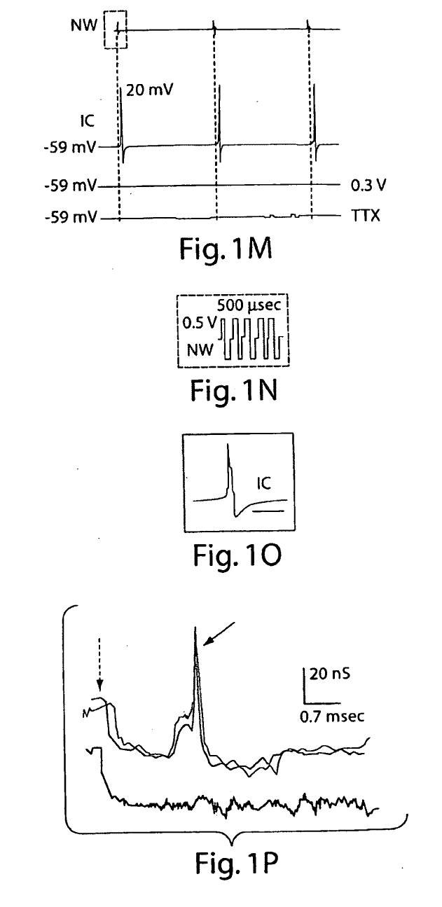

This example illustrates the preparation of certain nanowire/neuron devices, according to one embodiment of the invention. FIG. 1A is a general schematic for the preparation and assembly of oriented p- and/or n-type silicon nanowires in an aligned neuron/nanodevice array, with interconnection into well-defined FET device array structures, patterning of polylysine as an adhesion and growth factor to define neuron cell growth with respect to the device elements, and neuron growth under standard conditions (discussed in detail below). This approach is flexible allowing for variations in the addressable nanowire device separations down to at least 100 nm and device array geometry, incorporation of electronically distinct p- and n-type elements in well-defined positions, and/or variation in the number and spatial location of the hybrid nanowire/neuron junctions or synapses with respect to the cell body and neurite projections. Moreover, new chips incorporating such changes can be rapidly prototyped in about 1 day (from blank substrate to stage of neuron growth), which is an advantage compared to traditional planar FET structures, and allows for the rapid exploration of new ideas (or new integrated hybrid structures).

Optical images of an array of a repeating 1-neuron/1-nanowire motif with the soma remote and the axon directed across the respective nanowire element (FIGS. 1B-1F) show a high yield of 1:1 hybrid live cell devices with selective growth of the axon verified by marker specific fluorescence labeling and multicolor confocal microscopy. FIGS. 1B and 1C show optical images of growth-directed cortex neurons on a Si-nanowire array showing the reproducibility and high yield of the ‘axon-nanowire’ crossed-configuration. FIG. 1D is an optical image of a cortex neuron aligned across a nanowire device and FIG. 1E is a zoom-in of the box in FIG. 1D, which is the area where the axon crosses the nanowire. FIG. 1F is a confocal fluorescence image for a dual color labeled cortex neuron after 4 days in culture. The “tail” section (highlighted with the lower arrow) represents the growing axon labeled with axon-specific Tau protein antibody and the “head” section (highlighted with the upper arrow) represents a dendrite labeled with anti-MAP 2 antibody.

Analysis of these and additional chips indicated yields in excess of 90%, where clean patterning of polylysine and attachment of isolated live cells (see below for details) were found to be the most important of the determining factors. This high yield of completed hybrid structures is important to the ability to take advantage of flexible nanowire fabrication to build a range of distinct types of devices. Notably, the typical active junction area for devices, about 0.01 to 0.02 micrometer², is orders of magnitude smaller than microfabricated electrodes and planar FETs. The small hybrid junction sizes, which are similar to natural synapses, may offer important advantages for spatially-resolved detection of signals without complications of averaging extracellular potential changes over a large percentage of a given neuron, for integration of multiple hybrid elements on a single cell, and may yield good signal-to-noise since the nanowire is tightly coupled to the neuron through a thin, about 2 nm thick oxide over a substantial fraction of its active length.

Electrical communication was assessed in the neuron-nanowire structures by eliciting action potential spikes using a conventional glass microelectrode as a control impaled at the soma while simultaneously recording the intracellular potential and conductance at the microelectrode and nanowire FET, respectively. FIG. 1G shows the direct temporal correlation between the potential spikes initiated in the soma and the corresponding conductance peaks measured by the nanowire at the axon-nanowire junction. Expanded plots of single peaks in FIG. 1H exhibit shapes characteristic of neuronal action potentials. In FIG. 1G, the intracellular potential of an aligned cortex neuron (after 6 days in culture) was measured during stimulation with a 500 msec long current injection step of 0.1 nA; FIG. 1H shows the action potential measured intracellularly (“IC”). FIG. 1I is a graph showing a time-correlated signal from the axon measured using a p-type silicon nanowire device, while FIG. 1J is a graph of an action potential measured extracellularly by a p-type silicon nanowire device (“NW”). The direct correlation of the nanowire conductance peak with intracellular (“IC”) potential peak is expected for a p-type nanowire (these devices) since the relative potential at the outer membrane becomes more negative and then more positive (opposite to the measured IC potential) causing an accumulation of carriers/enhanced conductance and depletion/reduced conductance, respectively. Correspondingly, n-type nanowire elements may give inverted peaks or complementary response (see below).

FIG. 1K shows intracellular (upper) and nanowire (lower) electrical responses recorded for the same system as panels FIG. 1G and 1I, after severing the axon in contact with nanowire by using a micropipette. FIG. 1L shows intracellular (upper) and microfabricated electrode (lower) electrical responses of an aligned cortex neuron recorded using a device that did not contain a nanowire bridging the metal electrodes

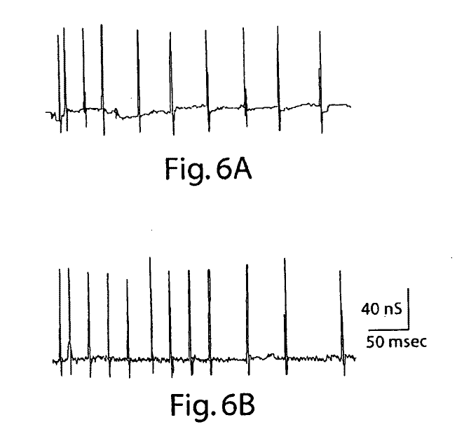

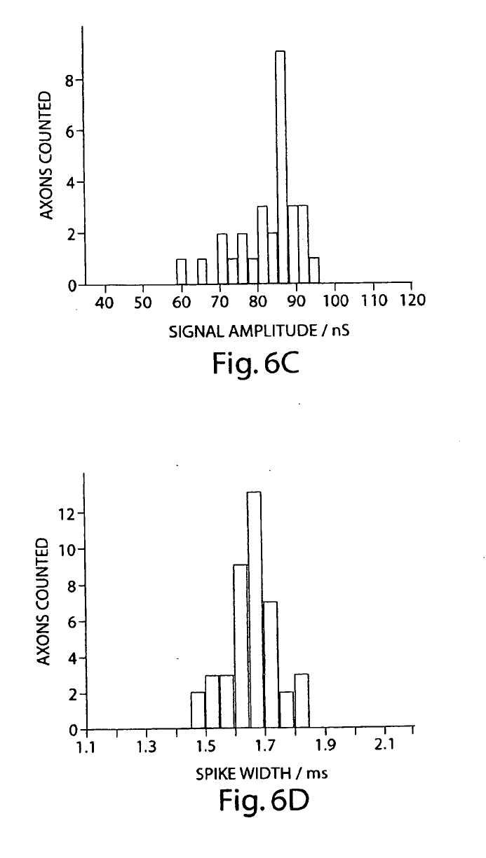

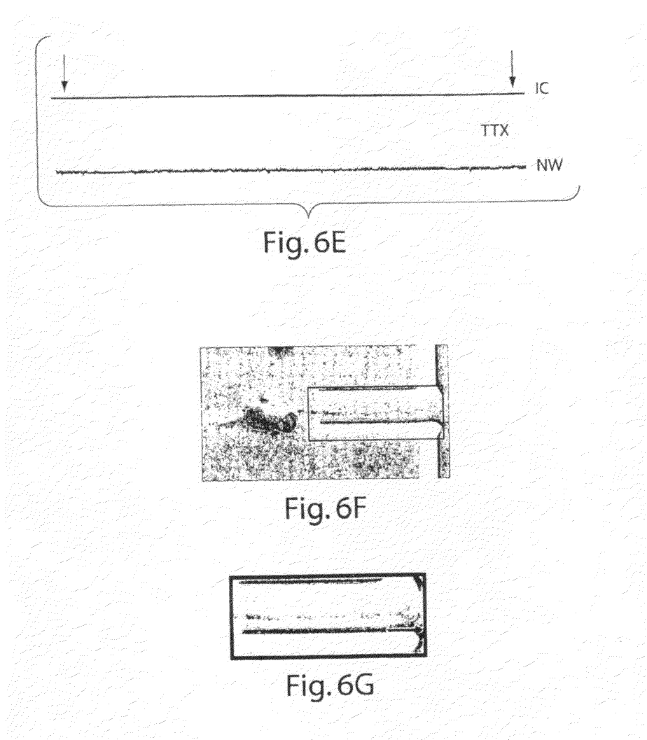

Several control experiments were performed to demonstrate that the nanowire conductance spikes corresponded to direct and localized detection of the action potential propagating along the neuronal axons, and are not due to artifacts. First, IC stimulation of higher frequency action potential spikes (FIGS. 6A-6B) also showed a direct temporal correspondence between the potential spikes initiated in the soma and the corresponding conductance peaks measured by the nanowire with a well-defined spike width and amplitude (FIGS. 6C-6D). The measurements in FIGS. 6A-6B were made on the same device/cell as in FIGS. 1G-1J. The nanowire detected action potentials for higher current intracellular stimuli of 0.3 nA (FIG. 6A) and 0.6 nA (FIG. 6B). FIGS. 6C and 6D show histograms of the spike amplitude (FIG. 6C) and width (FIG. 6D) measured from axons of aligned cortical neurons using p-type silicon nanowire devices.

Second, FIGS. 6F-6G showed that no conductance spikes are detected by the nanowire after severing the axon prior to the nanowire/axon junction. Third, no conductance spikes were observed in the same axon/electrode geometry (FIG. 1L, FIG. 6F-6G) when the nanowire element was absent, and fourth, after blocking voltage-dependent sodium channels with tetrodotoxin (TTX, FIG. 6E), no spikes were detected with either the IC glass microelectrode or at the nanowire/axon junction. FIG. 6E shows intracellular (upper trace) and extracellular (lower trace) nanowire electrical responses recorded for the same system as in FIG. 1G-IJ after bath addition of 0.5 μM TTX. Small black arrows indicate start/end of intracellular stimulation pulse (0.3 nA). FIGS. 6F-6G are optical images of a cortical neuron grown on a patterned substrate. FIG. 6G is a higher resolution image of the axon passing between microfabricated electrodes without a nanowire element (box in FIG. 6F).

Taken together, these results demonstrated that an intact and functional neuron with axon/nanowire junction was required to observe conductance spikes in the nanowire, and that electrical coupling between stimulation electrode or action potential spikes and the fabricated electrodes used to contact nanowires did not yield this behavior.

### EXAMPLE 3

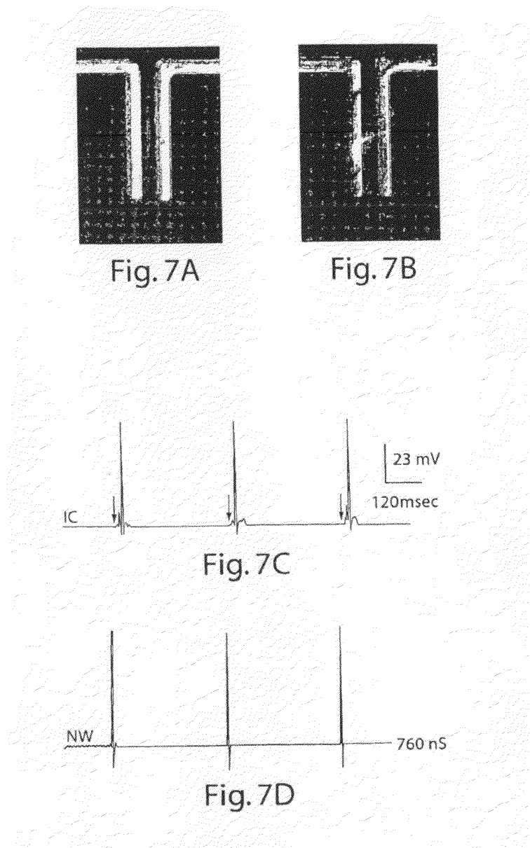

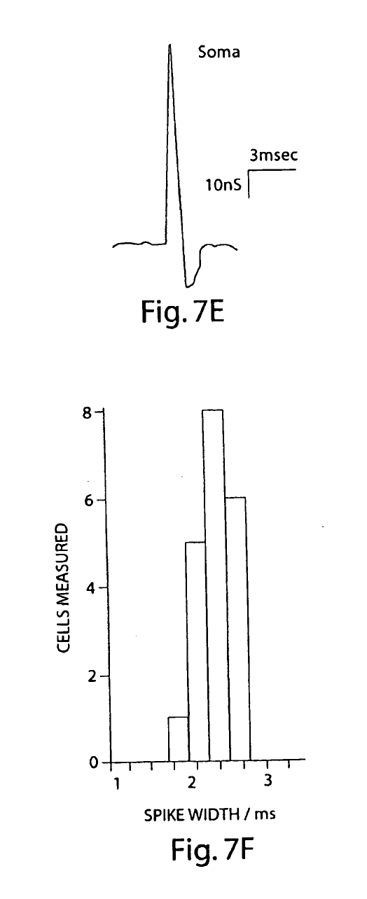

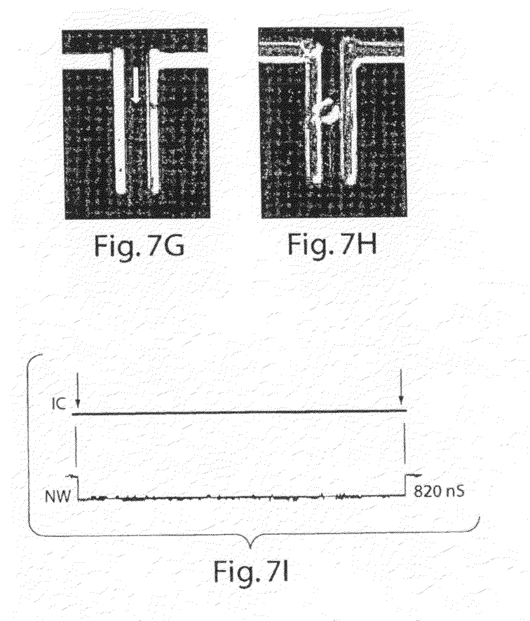

In this example, nanowire/soma and nanowire/dendrite hybrid structures were also investigated and found to exhibit excellent electrical communication. Intracellular stimulation of action potential spikes in the soma yielded correlated conductance peaks measured by the nanowire in a nanowire/soma structure (FIGS. 7A-7D). These figures show the silicon nanowire device before (FIG. 7A) and after (FIG. 7B) deposition and growth of a cortical neuron with the cell body over the nanowire device; the arrows highlight the positions of the nanowire and cell body, respectively. Also shown are intracellular (FIG. 7C) and nanowire (FIG. 7D) electrical responses of the neuron after intracellular current injection (arrows; 15 msec, 0.6 nA pulse).

The shape and width of the conductance peak (FIGS. 7E-7F) were similar to those determined from nanowire/axon structures. FIG. 7E is a graph showing representative electrical signals detected by nanowire devices for individual soma-nanowire connections. FIG. 7F is a histogram of the signal width recorded for soma/nanowire interfaces.

Control experiments further showed that no signals were detected in a nanowire following IC stimulation of an overlapping dead neuron (FIGS. 7G-7I), or following stimulation of an adjacent neuron that had no overlap with the nanowire. FIGS. 7G-7H are optical images of a nanowire device before (FIG. 7G) and after (FIG. 7H) the deposition of a neuron that had died. FIG. 7I shows intracellular (upper) and extracellular-nanowire (lower) electrical responses recorded after intracellular stimulation of this neuron.

These cell body measurements were similar to other studies of much larger planar FET/neuron devices, although the signal to noise with these nanowire devices was substantially better and comparable to the IC microelectrodes. Notably, it was also possible to assemble single dendrite/nanowire hybrid junctions and record with excellent signal-to-noise the propagation of spikes in the individual dendrites. This advantage in spatially-resolved measurements using multi-nanowire/neuron devices is discussed in more detail, below.

### EXAMPLE 4

In this example, nanowire devices were used to stimulate neuronal activity through nanowire/axon junctions. These hybrid junctions are interesting in that the size scale is close to natural dendrite/axon synapses, and thus it is reasonable to consider them as artificial synapses; in contrast, larger microfabricated structures used previously for neuron stimulation are on a very different (orders of magnitude larger) size scale. Application of biphasic excitory pulse sequences to the nanowire of nanowire/axon junctions (FIG. 1M) results in detection of somatic action potential spikes. With this biphasic pulse sequence IC potential spikes were observed in 86% of the stimulation trials. The excitation of action potential spikes also showed a threshold of about 0.4 V, where no potential spikes were observed with the IC electrode when driving the nanowire below this value (FIG. 1M). In addition, nanowire stimulation above threshold of hybrid structures treated with TTX (FIG. 1M) did not show IC potential spikes, thus showing that electronically functional neurons were required to observe this behavior.

In FIG. 1M, the intracellular electrical recording of a cortex neuron after axon-nanowire stimulation is shown (trains of five rectangular biphasic-type stimuli, 500 microsecond width) (upper curve). Electrical stimuli curve and amplification of the stimuli section are also shown (dashed box; FIG. 1N). The lower three curves are intracellular recording after nanowire stimulation using rectangular biphasic-type stimuli of: (upper) 0.5 V stimuli amplitude, (middle) 0.3 V amplitude and (lower) after bath application of 0.5 micromolar TTX and 0.5 V stimuli amplitude. One curve has been expanded (FIG. 1O).

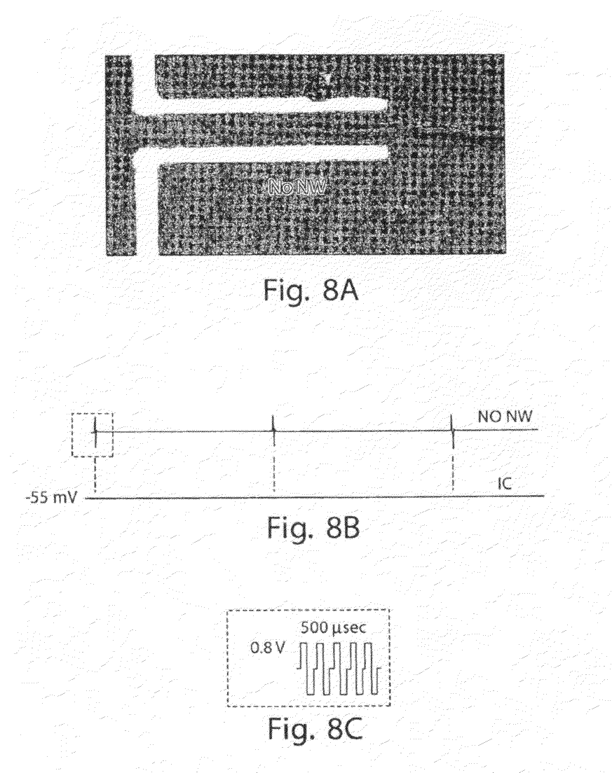

The role that the nanowire/axon junction played in stimulating action potentials was further demonstrated by making measurements on similar structures in which the key nanowire element was missing (FIG. 8A); stimulation of these structures (above threshold for nanowire/axon junctions) led to no observable IC somatic potential spikes (FIG. 8B).

FIG. 8A is an optical image of the axon from a cortical neuron passing between microfabricated electrodes without a nanowire. Neuron growth was directed using a polylysine pattern similar to that shown in FIG. 1A. FIG. 8B is a graph showing the electrical output (stimulation curve) from the microfabricated electrodes (upper trace) and corresponding intracellular signal (lower trace) following stimulation pulse sequence applied to the electrodes. The specific pulse sequence is shown in the dashed rectangle (FIG. 8C). Thus, the highly localized excitation possible with nanowires coupled with potential for multiple inputs enables interesting opportunities for both fundamental neurobiology studies and hybrid electronics.

Along these lines, it is noted that the stimulation and detection capabilities of a single nanowire can be exploited in a single experiment. Specifically, reconfiguration of the nanowire to FET measurement following stimulation (FIG. 1P) showed that the elicited action potential can be recorded as a conductance spike with good signal-to-noise and about a 1 msec delay from the stimulation train when excitation is above threshold. FIG. 1P shows a nanowire recorded electrical responses after axonal stimulation using the same nanowire as stimulating agent (upper curves). Trains of five rectangular biphasic-type stimuli (train width 500 microseconds) were applied to the recording nanowire. The lower curve corresponds to the nanowire-recorded electrical response after application of a stimuli train of five rectangular pulses of lower amplitude, 0.3 V, and equal duration as before. The solid arrow corresponds to the neuronal signal and the dashed arrow corresponds to the coupling of stimulation pulse to the device output.

### EXAMPLE 5

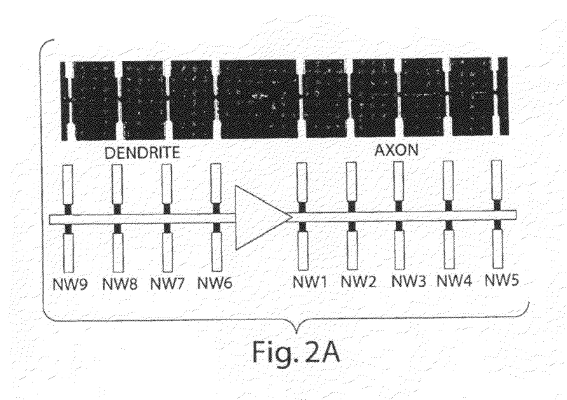

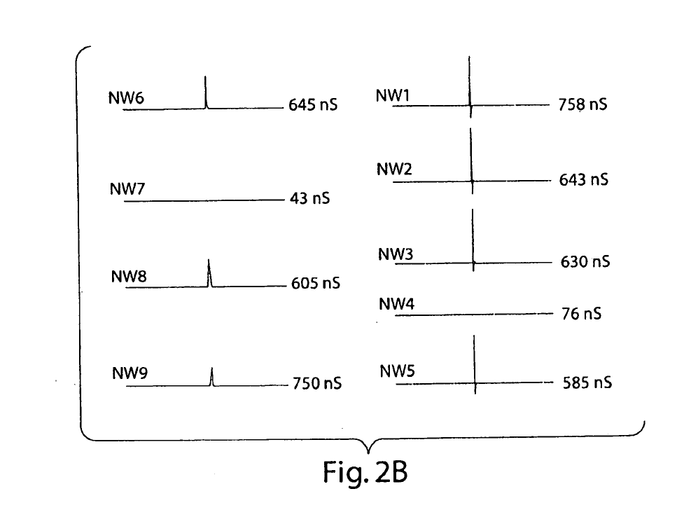

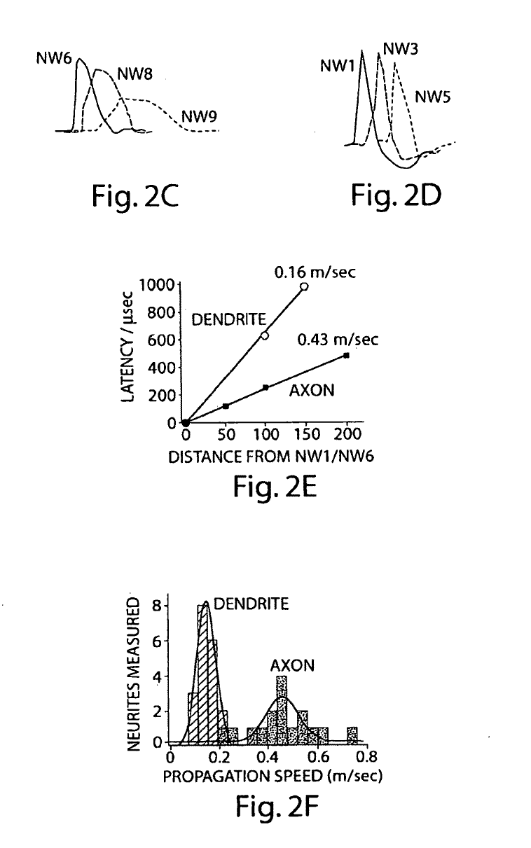

This example illustrates the flexibility of this approach to assemble and characterize hybrid nanowire/neuron devices in which the number and spatial arrangement of nanowires interfaced to the neurons are varied. First, a device structure consisting of a linear array of 4-nanowire FETs, a gap, and 5-nanowire FETs was designed (FIG. 2A) to test whether simultaneous and temporally-resolved propagation and back propagation of action potential spikes in axons and dendrites, respectively, could be detected. FIG. 2A is an optical image of a cortex neuron with axon and dendrite aligned in opposite directions and (bottom) corresponding schematic diagram.

By employing the polylysine patterning introduced above (see below) optical images (FIG. 2A) demonstrated well-defined growth of rat cortical neurons with the cell body localized in the gap with an axon and dendrite guided in opposite directions across the two linear FET arrays. The specific polarity of growth (e.g., axon across the 4 or 5-FET array) was not controlled, but was readily identified by the faster growing projection (the axon) during culture, and subsequently by electrical response (see below) and post measurement fluorescent imaging. On a given chip, about 20 of the repeating nanowire array structures were fabricated, and following low density neuron adsorption/growth obtain a yield of about 80% hybrid structures/chip.

These multi-nanowire/neuron arrays were characterized by simultaneous detection of the conductance output from nanowires following IC stimulation at the soma. FIG. 2B shows electrical responses measured from dendrite/nanowire devices (left traces, NW 6-9) and axon / nanowire devices (right traces, NW 1-5) after intracellular stimulation with a 15 ms, 0.5 nA current pulse. It was found that stimulation of action potential spikes in the soma yielded correlated conductance peaks in nanowire elements forming the nanowire/axon and nanowire/dendrite junctions (FIG. 2B). Qualitatively, these data demonstrate several key points. First, seven of the nine independently addressable nanowire/neurite junctions yielded reproducible conductance spikes correlated with IC stimulation. Higher yields of functioning elements have also been achieved (see below), although this about 80% yield still left three and four spatially-defined local detectors on the dendrite and axon, respectively. It is believed that this level of integration of hybrid electronic/biological synapses is unique to this work. Second, the conductance spikes recorded along the axon by elements 1-5 maintained sharp peak shape and relatively constant peak amplitude. In contrast, the conductance spikes measured by elements 6-9 along the dendrite exhibited noticeable broadening and reduced amplitude.

Signals from the multiple, spatially-separated nanowire/neurite junctions were recorded simultaneously, and thus enabled spike propagation to be quantified in both axons and dendrites. A comparison of high-resolution conductance-time data (FIGS. 2C-2D, which are expansions of peaks from FIG. 2B, elucidating the evolution of peak shape as it propagates along each process) demonstrated that the propagation delay of spikes in the dendrite and axon following initiation in the soma could be readily resolved, and moreover, showed a clear peak reduction and temporal spreading in the dendrite and little change in the axon over distances of about 200 micrometers in each. These latter observations were consistent with passive and active propagation mechanisms, respectively.

By using the first nanowire (i.e., NW1 and NW6) in each neurite as reference, (FIG. 2E) signal propagation rates of 0.16 m/sec for dendrites and 0.43 m/sec for axons were calculated. In trials with different neurons, it was found that these rates had Gaussian distributions of 0.15±0.04 m/sec and 0.46±0.06 n/sec for dendrites and axons respectively (FIG. 2F); these data were comparable to reported propagation rates measured by conventional electrophysiological and optical methods. FIG. 2E is a plot showing latency time as a function of distance from NW1 and NW6 for axons and dendrites, respectively; FIG. 2F is a histogram of propagation speed through axons and dendrites. Indeed, the high-sensitivity, “multi-site” electrical recording of neuronal activity and signal propagation has similarities to optical methods, which rely on the injection of voltage-sensitive dyes, but also possesses important advantages. For example, it was possible to achieve substantially higher resolution (at least to the 100 nm level) by changing the device separation in arrays. In addition, the nanowire elements could be assembled into structures capable of probing simultaneously multiple individual neurites, which is not currently possible with other tools, and also use one or more of the nanowire/neurite “synapses” as inputs to initiate and/or modulate signal propagation.

### EXAMPLE 6

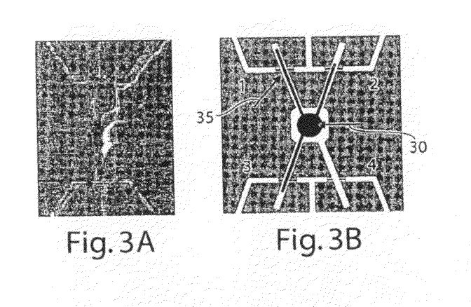

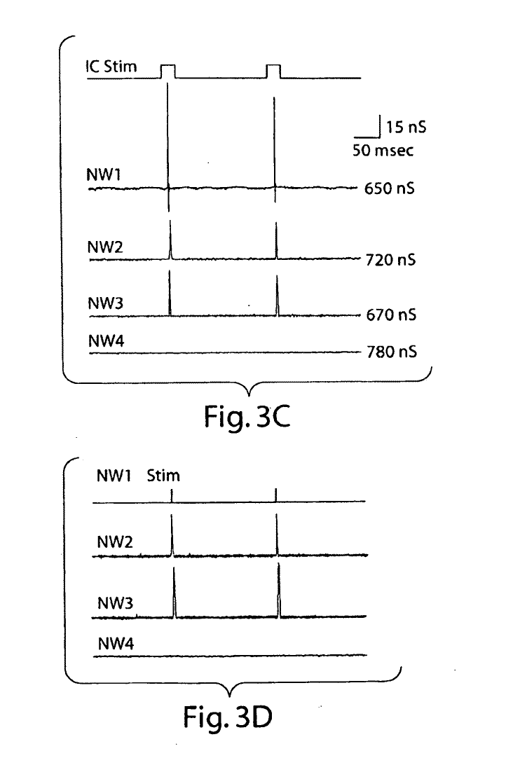

To explore further the potential of multi-nanowire/neurite artificial synapses, in this example, hybrid structures were assembled (FIGS. 3A and 3B) having a central cell body and four peripheral nanowire elements arranged at the comers of a rectangle with patterning designed to promote neurite growth across these elements. A representative optical image of a cortex neuron connected to 3 of the 4 functional nanowire devices in the array (FIG. 3A) verified this basic motif with hybrid nanowire/axon, and two nanowire/dendrite elements at positions 1, 2 and 3, respectively. FIG. 3B is a schematic showing two possible stimulation approaches: intracellular stimulation (arrow 30 in soma) and extracellular nanowire-based stimulation (arrow 35 on NW1).

FIG. 3C shows traces of intracellular current stimulation (15 msec current injection pulses of 0.5 nA) and resulting nanowire (NW1, NW2, NW3 and NW4) electrical responses. Note that NW4 is not electrically connected to any section of the neuron and thus functions as an internal control for all the experiments. This figure showed that stimulation of action potential spikes in the soma yields correlated conductance peaks in the nanowire/axon (NW1) and nanowire/dendrite (NW2, NW3), while no signal was observed in a good detector (NW4) that had no visible neurite overlap. In addition, NW1 was used, which forms an electrical junction with the axon, as a local stimulatory input to elicit action potential spikes that were subsequently detected in the two dendrites crossing elements 2 and 3. The lack of observed signal from NW4 demonstrates the absence of cross-talk in these hybrid devices. FIG. 3D shows traces of pulses (trains of five rectangular biphasic-type stimuli, train width 500 microseconds) applied to NW1 for antidromic stimulation of neuron. The response was measured by the dendrite/nanowire junctions at NW2 and NW3. No neural connection was present on NW4, which serves as control

These basic studies were interesting both in their potential application for neurobiology, for example mapping in detail spike propagation and the influence of artificial synapses (inputs) at the single neuron and network level, and as hybrid circuit elements that could be used for logic and ultimately information processing.

### EXAMPLE 7

These multi-nanowire/neurite hybrid structures and electrical data suggest extensions to more complex input/output configurations and/or circuit elements with corresponding potential for greater functionality, as is shown in this example.

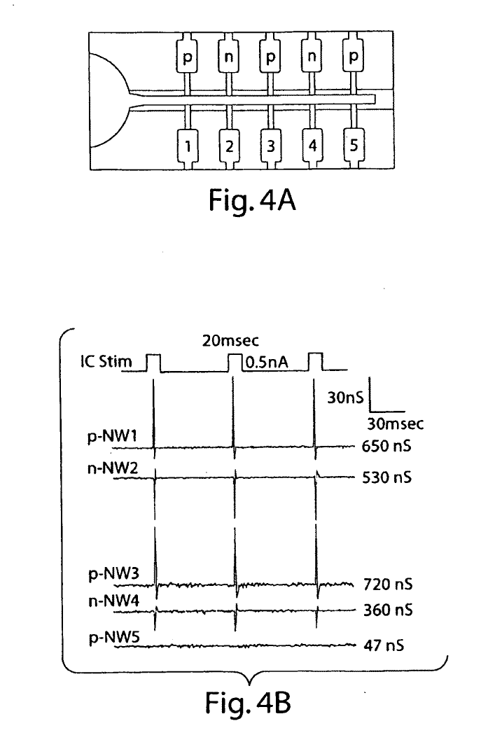

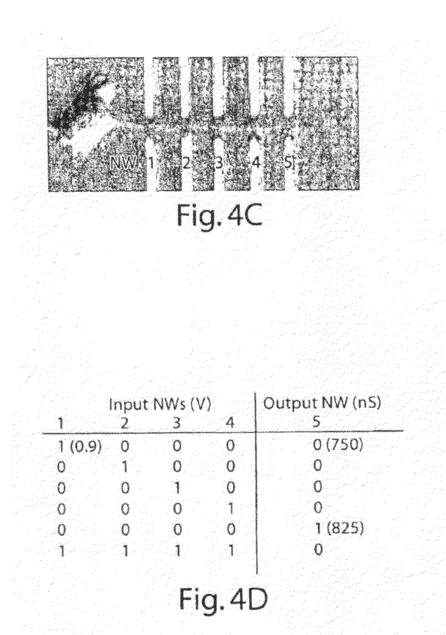

First, the flexibility of this nanowire assembly approach was used to fabricate rationally hybrid device arrays consisting of alternating p-type and n-type nanowire elements that form sequential junctions with the axon of neurons (FIG. 4A). FIG. 4A is a schematic of an aligned axon crossing an alternating array of five p- and n-type nanowire devices. The spacing between the devices was 10 micrometers. An obvious motivation for exploring this more complex arrangement of nanoelectronic elements rests on the importance that complementary signals, which are produced with p- and n-type materials, have in digital electronics and computing. Notably, IC stimulation of action potential spikes in the soma yielded temporally correlated, alternating conductance peaks/dips in nanowire elements progressively from NW1 to NW4. FIG. 4B shows traces of intracellular current stimulation (20 msec pulses of 0.5 nA amplitude) and resulting signals measured by the p-type and n-type devices depicted in the preceding schematic (FIG. 4A).

These results were consistent with gating of the p- and n-type nanowires by the change in membrane potential associated with the propagating action potential, and showed that it was possible to generate complementary signals in the hybrid structures. While the complementary signals were illustrated as variations in conductance, complementary output voltages could also be produced in current biased devices. It is believed that the relative ease of assembling complementary nanowire/neuron hybrid devices in different structural motifs makes this a rich area for device and circuit concepts drawn from digital electronics, as well as novel processing strategies where the complementary nanowire signals are used as inhibitory and excitatory inputs for artificial synapses.

### EXAMPLE 8

Explored in this example were hybrid nanowire/neuron arrays as logic gates, where several nanowire/axon junctions were configured as inputs and one of the hybrid junctions was used as an output. The inputs or artificial synapses modified signal propagation and yielded a well-defined logic state at the output. As an example, hybrid structures of five independent nanowire/axon elements were characterized (FIG. 4C), where the first four are inputs with each nanowire were set at a controllable potential, and the last junction (NW5) detects the output state. When the inputs were set low (no applied voltage), a high value was detected at NW5 following stimulation of an action potential spike. On the other hand, if any of the input nanowires was set high (0.9 V), a low signal was detected in NW5 following stimulation of a spike. FIG. 4C is an optical image of a cortex neuron with its axon aligned on an array of five p-type nanowire devices. Nanowires 1-4 acted as inputs that would either inhibit the propagation of a signal by hyperpolarizing the membrane (‘1’) or allow it to pass (‘0’). The output, measured by nanowire 5, represented the presence (“1”) or lack (“0”) of signals that were elicited intracellularly or elsewhere. These results are summarized in the form of a truth table (FIG. 4D), which showed that this hybrid structure functions analogous to a 4-input NOR (not OR) logic gate.

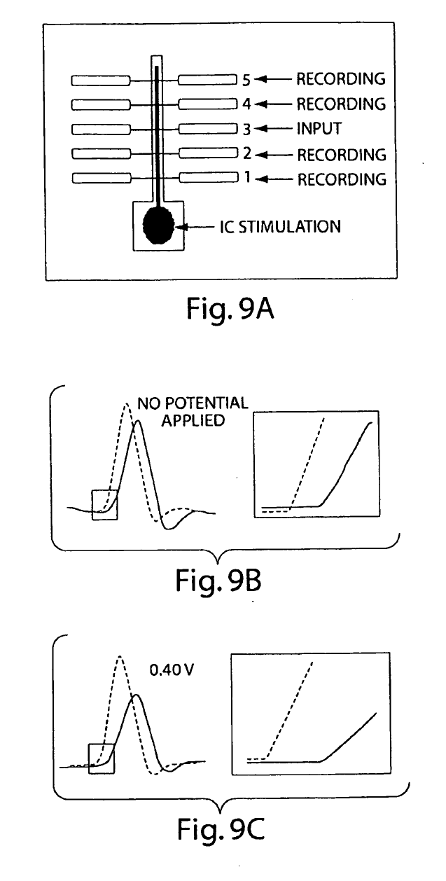

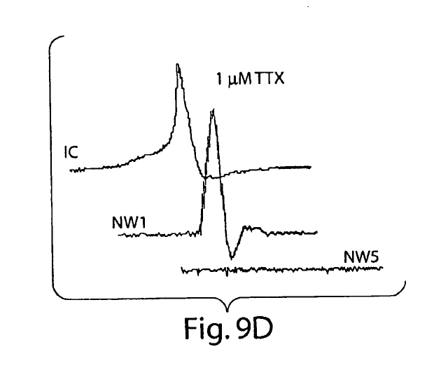

The mechanism underlying the behavior of this hybrid NOR logic gate is believed to be local anodic hyperpolarization of the membrane at nanowire/axon synapses, when the nanowire voltage was set high. This hyperpolarization can block the propagation of action potential spikes, and can result in a low output (i.e., no spike). Additional studies were performed to explore the scope of controlling input/output in these hybrid circuit elements as this suggests other ways of performing logical operations (FIG. 9). First, applying input potentials less than high value required to block spike propagation, resulted in a reduction in the measured propagation speed and spike amplitude. These results have some analogy to synaptic modulation of signal propagation in neuronal networks, as well as analog electronics. FIG. 9A is a schematic illustrating the structure of the multi-nanowire/neuron device. The structure is similar to optical image in FIG. 4C. FIGS. 9B-9C shows electrical signals recorded at NW1 and NW5 before (FIG. 9B) and after IC stimulation (FIG. 9C); the applied (hyperpolarizing) pulse of 0.4 V was applied to NW3.

Along these lines, inputs for the hybrid nanowire/neuron circuit elements, as in biological neuronal networks, were not limited to electrical inputs, but could be chemical as well. FIG. 9D shows IC and nanowire output signals recorded after focal application of 0.5 micromolar TTX to the axon section between NW3-NW4. The nanowire signals were recorded before (NW1) and after (NW5) the injection point of the TTX. Local release of TTX at the same input nanowire as above (NW3) blocked the spike propagation and resulted in low output at NW5. Although TTX was injected in these experiments, it is possible to release neurotransmitters, channel blockers and other chemicals selectively using chemically-derivatized nanowires, thereby enhancing the modes of input/signal modulation and output in these hybrid nanoelectronic devices.

### EXAMPLE 9

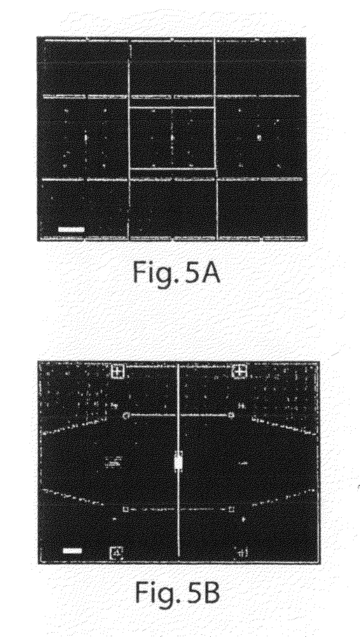

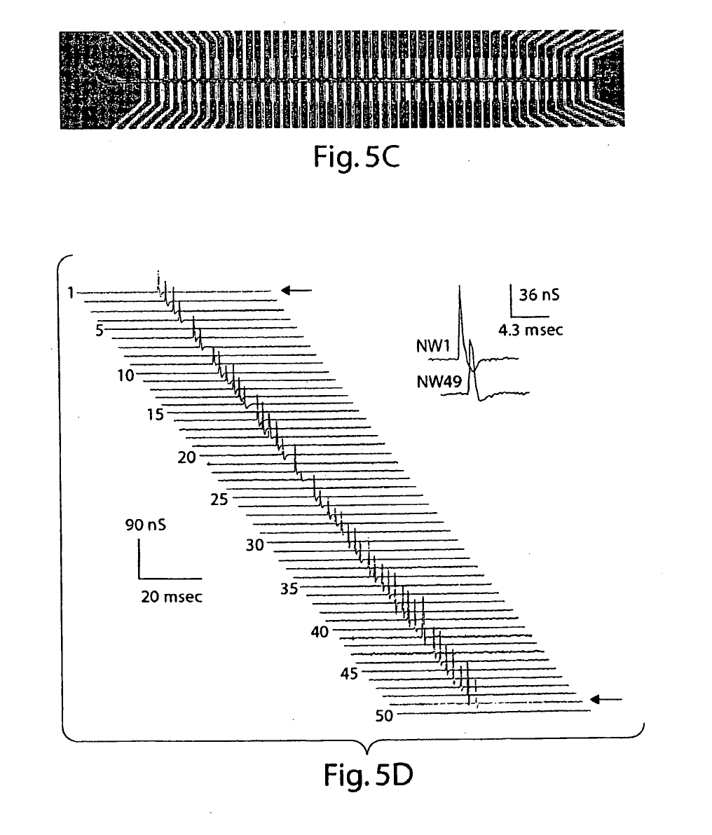

This approach can be readily extended to highly integrated systems that could open up opportunities in a number of areas. To demonstrate this idea a repeating structure was designed and fabricated (FIGS. 5A-5B) that had 50 addressable nanowire elements per neuron. This structure was chosen to show the capability of single cell hybrid structures at much higher density of nanoelectronics devices, but could be readily reconfigured, for example, into structures with different geometries, nanowire device spacings, and/or multiple cells. FIG. 5A is an optical image of a chip having six device arrays of 50 nanowire elements each and associated metal interconnects; FIG. 5B is an optical image corresponding to the area enclosed by the blue rectangle and showing two 50 nanowire element arrays. The rectangle highlights an area representative of the hybrid device array shown in FIG. 5C. The scale bars are 5 and 1 mm, respectively.

FIG. 5C is an optical image of aligned axon crossing an array of 50 devices with 10 micrometer interdevice spacing, showing that well-aligned neuron growth was achieved over these large nanowire device arrays using polylysine patterning, and electrical transport measurements made after neuron growth demonstrates a high yield of good nanowire FET devices: 43/50 devices with conductance values from 550 to 870 nS. The yield of functional devices was 86%.

IC stimulation of action potentials in the soma yields a mapping of the spike propagation by the 43 working devices over the about 500 micrometer long axon (FIG. 5D). The peak latency from NW1 to NW49 was 1060 microseconds. These data exhibited little decay in peak amplitude from NW1 to NW49, which is consistent with the active propagation process. More importantly, these data demonstrate an unprecedented density of artificial “electrical synapses,” each which could be independently monitored or stimulated, and thus provide a clear indication of promise future for hybrid processing circuits.

### EXAMPLE 10

This example describes certain protocols and methods that may be useful in various embodiments of the invention.

Nanowire array fabrication. The 20 nm diameter silicon nanowires were synthesized by gold nanocluster chemical vapor deposition as described previously. Diborane and phophine with B:Si and P:Si ratios of 1:4000 were used to prepare p-type and n-type nanowires, respectively. Nanowires were aligned on oxidized surface of silicon chips (600 nm thick oxide, NOVA Electronic Materials, Ltd.) using flow-directed or Langmuir-Blodgett techniques, where a uniform parallel nanowire array with controlled separation could be prepared over entire chip with the latter method. Source and drain contacts to the nanowires were defined following assembly using reported photolithography and metal deposition (60 nm Ni), and were passivated prior to resist lift-off by deposition of an about 150 nm thick Si₃N₄ by plasma-enhanced CVD.

Chip surface patterning. A second photolithography step was used to define patterns of 30-50 micrometer squares (for attachment of cell bodies) and 2-3 micrometer wide lines (for guided axon and dendrite growth) on chips containing fully fabricated nanowire FETs; the patterns were registered with respect to nanowire devices with about 1 micrometer accuracy. In brief, completed device chips were modified in a 1% (v/v) dichloromethane solution with (heptadecafluoro)-1,1,2,2-tetrahydrodecyldimethylchlorosilane (Gelest, Inc.) for 1 h, rinsed with dichloromethane and cured at 110° C. for 10 min. Following photolithography patterning using a positive photoresist (Shipley S1805), the fluorosilane in the exposed areas was removed by oxygen plasma (50 W for 5 minutes) and the chips were then soaked in an aqueous polylysine solution overnight (0.2-0.5 mg/ml, MW 70,000-150,000). The remaining photoresist was removed in a 30 minute acetone wash and the entire chip was sterilized by ethanol washes and a standard autoclave cycle.

Stage preparation. Fully patterned chips were mounted on a temperature-controlled microscopy platform and clamped beneath a plastic perfusion chamber. Pads without Si₃N₄ passivation extended beyond the perfusion chamber (i.e., were not in contact with the buffer solution during neuron incubation or measurement), were wire-bonded to a set of pin sockets affixed to either side of the platform. The mounted and wired chips were sterilized (1 autoclave cycle and 1 h UV-light), and then preincubated at 37° C. in 5% CO₂ before cell deposition.

Cell culturing. Day 18-19 embryonic primary cortical cells (and/or primary hippocampal cells) from Sprague/Dawley or Fischer 344 rat brain (99.9% glia-free, GTS Inc.) were suspended in culture medium (neurobasal serum-free medium containing 0.5 mM glutamine, B27 supplement and streptomycin antibiotic) and further diluted with neurobasal/glutamine/B27/streptomycin to the desired plating density. The cell suspension was transferred to the preincubated chip surfaces and incubated for 20-120 minutes at 37° C. in a 5% CO₂ incubator depending on the desired cell density. Excess cells were removed (except for a wetting layer on the chip surface) and fresh, pre warmed medium was added. Neuron chips were incubated at 37° C. with 5% CO₂ for periods of 4-8 days.

Electrophysiology. Intracellular stimulation/recording experiments were carried out in standard manner 2 using glass microelectrodes back-filled with 3 M potassium chloride, and contacted with a Ag/AgCl wire (resistance 25-70 megohms (MΩ)); the electrodes were mounted on motorized 3-axis micromanipulators (DC-3K, Märzhäuser Wetzlar GmbH & Co.), and controlled using standard amplifier-bridge electronics (IE-210, Warner Instrument Corp.) under computer control. All measurements were carried out at 37° C. with the chip surface submerged in an electrophysiology bath solution containing 145 mM NaCl, 3 mM KCl, 3 mM CaCl₂, 1 mM MgCl₂, 10 mM glucose and 10 mM HEPES, pH 7.25. Rest membrane potentials were estimated after entering in a whole cell configuration, and action potentials were typically elicited by brief (0.3-0.5 ms) or long (500 ms) depolarizing currents of 0.3-0.9 nA. In some experiments, tetrodotoxin (TTX) (0.5-1 micromolar, Sigma) was introduced at specific locations using a pico-injector (PLI-100 Plus Pico-Injector, Medical Systems Corp.) or globally through bath application. All microelectrode and injection steps were made under direct optical observation, and data were recorded and processed using LabScribe.

Nanowire-device measurements. All studies were carried out at 37° C. with the chip surface submerged in an electrophysiology bath solution (see above). The nanowire FET conductance was measured in AC mode (1-50 kHz; 30 mV peak-to-peak) with the DC bias set to 0 V; the signal was amplified with a variable gain preamplifier (1211 current preamplifier, DL Instruments Inc.) and detected using a lock-in amplifier (DSP dual phase lock-in, Stanford Research Systems). The output data was recorded using an AID converter at 10 or 100 kSa/s. Nanowire-based stimulation was carried out by applying biphasic square wave pulses (amplitude 0-1 V) while inhibition/hyperpolarization was achieved by applying potential steps (amplitude 0-0.2 V). In both cases, the signal was applied to source and drain electrodes simultaneously.

Immunohistochemistry. Axons and dendrites were identified following experiments by selective antibodies using monoclonal rabbit axon-specific Tau protein antibody (1:1000 dilution, Chemicon Inc.; rabbit anti-synapsin I antibody was also used for axon labeling) and monoclonal mouse antibody MAP-2 (1:500 dilution, Chemicon Inc.), respectively.

Neurons were fixed with 4% formaldehyde in PBS for 40 min at 4° C., permeabilized with 0.25% Triton X-100 for 5 min and rinsed three times for 5 min with PBS. After treating with preblock-buffer (0.05% Triton-X, 5% fetal bovine serum in PBS) for 2 hours at 4° C., cultures were incubated with the primary antibodies overnight in the dark at 4° C. In the double labeling experiments, the two primary antibodies were incubated together. Fluorophore-conjugated secondary antibodies for axons (AlexaFluor-546 anti-rabbit IgG) and dendrites (Fluorescein anti-mouse IgG) were conjugated prior to imaging with a confocal microscope system (LSM 510 Meta, Zeiss). Controls without primary antibodies and single-labeled samples were also carried out to verify the interpretation of the double-label experiments. In most cases examined, it was found that the first projection extending from the neuron body along a patterned polylysine-patterned line is an axon (>80% cases, during the first 2-3 days in culture).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. (canceled)

2. The article of claim 10, further comprising electrical circuitry constructed and arranged to pass the electrical current through the nanoscale wire.

3. The article of claim 10, wherein the cell is in physical contact with the nanoscale wire.

4. The article of claim 10, wherein the cell is a neuron.

5. The article of claim 10, comprising at least 10 nanoscale wires each in electrical communication with the cell.

6-7. (canceled)

8. The article of claim 10, wherein the nanoscale wire is a nanotube.

9. The article of claim 10, wherein the nanoscale wire is a semiconductor nanowire.

10. An article, comprising:
a nanoscale wire positioned between two electrical connectors each on a surface; and
a cell in electrical communication with the nanoscale wire such that an electrical state of the cell can be altered by passing electrical current through the nanoscale wire.

11. The article of claim 10, wherein the cell is in electrical communication with a field-effect transistor, the field-effect transistor comprising the nanoscale wire.

12. The article of claim 10, wherein the article is a logic gate.

13-14. (canceled)

15. The article of claim 10, further comprising a sensing electrode in electrical communication with the cell.

16-19. (canceled)

20. A method, comprising:
passing electrical current through a nanoscale wire in physical contact with a neuron; and
passing sufficient electrical current through the nanoscale wire such that the neuron depolarizes.

21. The method of claim 20, further comprising exposing the neuron to a chemical species suspected of being able to alter an electrical state of the neuron.

22. The method of claim 20, further comprising recording an electrical response in the neuron due to the electrical current.

23. (canceled)

24. A method, comprising:
determining an electrical state of a cell by passing current through a nanoscale wire positioned between two electrical connectors each on a surface.

25. The method of claim 24, wherein the cell is a neuron.

26. The method of claim 25, comprising determining an electrical state of an axon of the neuron.

27. The method of claim 25, comprising determining an electrical state of a dendrite of the neuron.

28. The method of claim 25, comprising determining an electrical state of a soma of the neuron.

29. The method of claim 25, comprising determining an action potential of the neuron using the nanoscale wire.

30. The method of claim 24, comprising recording the electrical state of a cell using a plurality of nanoscale wires.

31-54. (canceled)
